SEQUENCE LISTING (10 sequences)

SEQ ID NO: 1 - DNA, 717 bp, synthetic construct
  source 1..717: mol_type = other DNA; note = GFP
  CDS 1..717
atgtctaagggcgaagagcttttcacgggcgtcgttcctatcctggtagagttggacggtgatgtgaatggccacaaattttctgtgtctggcgagggtgagggcgatgccacttatgggaaattgactttaaaatttatttgcacaacaggtaaattacctgtgccttggcctactctggttacgactttcgcgtatgggctgcaatgttttgcccggtatcctgaccatatgaagcagcatgacttctttaagagcgcgatgccagaaggttacgtgcaggaacgcactatcttctttaaagacgacgggaattataaaactcgggcggaggtaaaatttgagggggacacccttgtcaaccgcatcgaattgaaggggatcgacttcaaagaggacggcaacattcttgggcataagctcgaatacaattacaactcgcacaatgtctacattatggctgataaacagaaaaacggtatcaaggtaaatttcaagattcgccataatatcgaggatggttcagtccagttggctgaccactaccagcagaatacacctatcggggacggcccagtgttacttcctgacaaccattacttatcgactcagtctgctctgtctaaagacccaaatgaaaagcgggaccacatggtactgctggaatttgttaccgccgcagggattacacacgggatggatgaattgtataagtaa

SEQ ID NO: 2 - protein, 238 aa, synthetic construct
  source 1..238: note = GFP
MSKGEELFTGVVPILVELDGDVNGHKFSVSGEGEGDATYGKLTLKFICTTGKLPVPWPTLVTTFAYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 3 - DNA, 480 bp, synthetic construct
  source 1..480: mol_type = other DNA; note = folA
  CDS 1..480
atgattagtttaattgctgctctggcggtggatcgcgtgattggaatggaaaacgctatgccctggaatttaccggcggacctggcttggttcaagcgcaatacgttgaataaacctgttattatgggacgccatacctgggagtctattggacgcccccttccgggccgcaaaaacattattctttcgtcacaacctggcactgatgatcgcgtgacatgggtaaagagtgtcgatgaagctatcgccgcctgtggcgatgtcccagaaattatggtaattggcggagggcgcgtctacgagcaattccttcctaaggcccagaaactgtacttgacacatattgatgccgaggttgaaggtgacacccacttcccagattacgaaccggatgattgggaaagcgtttttagtgaatttcacgacgccgatgcccagaactctcactcctattgtttcgaaatcttagagcgtcgttaa

SEQ ID NO: 4 - protein, 159 aa, synthetic construct [sequence omitted]

SEQ ID NO: 5 - DNA, 384 bp, synthetic construct
  source 1..384: mol_type = other DNA; note = Anti-HER2 VHH
  CDS 1..384
atggaagtacaattagtagaatcagggggcgggttagtccaggctggggggtcgttgcggttgtcttgtgccacctccgggattactttcatgcgttatgctttgggctggtatcgccaatcgcctggtaaacagcgtgagatggtggcatcaattaatagcggtggcacaacgaactacgctgatagtgttaaaggtcgtttcaccatcagtcgggataacgcaaaaaataccgtgtacttgcagatgaactccttgaagcctgaagatactgctgtctattactgcaatgcgcggtgggtgaagccacagtttattgataacaactattggggtcaggggactcaggtgacggtcagcagccaccatcaccaccatcattaa

SEQ ID NO: 6 - protein, 127 aa, synthetic construct
  source 1..127: note = Anti-HER2 VHH
MEVQLVESGGGLVQAGGSLRLSCATSGITFMRYALGWYRQSPGKQREMVASINSGGTTNYADSVKGRFTISRDNAKNTVYLQMNSLKPEDTAVYYCNARWVKPQFIDNNYWGQGTQVTVSSHHHHHH

SEQ ID NO: 7 - DNA, 405 bp, synthetic construct
  source 1..405: mol_type = other DNA; note = sars-cov2 VHH
  CDS 1..405
atgcaagtacagttagtagagagtggtggcggtctcgtacaaccagggggctctctccgtttaagttgtgccgcttcgggtattacgttggattattatgccattggttggtttcggcaggcacctgggaaggagcgggagggtgtcagccgtatccggtcatccgatgggtctaccaattatgccgatagcgtcaaaggtcgttttaccatgtcgcgtgataacgcaaagaacacagtataccttcaaatgaactcattaaaaccagaagatacggctgtctactattgtgcatatggtccactgacgaaatatggctcaagttggtattggccttacgaatacgactactggggccaagggacgcaagtaactgttagtagccaccatcaccaccatcattaa

SEQ ID NO: 8 - protein, 134 aa, synthetic construct
  source 1..134: note = sars-cov2 VHH
MQVQLVESGGGLVQPGGSLRLSCAASGITLDYYAIGWFRQAPGKEREGVSRIRSSDGSTNYADSVKGRFTMSRDNAKNTVYLQMNSLKPEDTAVYYCAYGPLTKYGSSWYWPYEYDYWGQGTQVTVSSHHHHHH

SEQ ID NO: 9 - DNA, 711 bp, synthetic construct
  source 1..711: mol_type = other DNA; note = mCherry
  CDS 1..711
atggtcagcaaaggcgaagaggacaatatggcaatcattaaagaatttatgcgtttcaaggtgcacatggaaggtagcgttaacggtcacgaatttgagattgagggtgagggtgaaggtcgcccgtatgaaggcacccaaaccgcgaaactgaaggtcaccaagggtggcccgctgccgtttgcctgggacattctgagcccgcagtttatgtacggtagcaaggcgtacgttaagcatccggcagatattccggactatctgaagctgtcgttccctgagggtttcaaatgggagcgtgtgatgaatttcgaagatggcggcgtcgttacggtgacccaagactctagcctgcaagacggcgagttcatctacaaagtcaaactgcgcggtaccaactttccaagcgatggtccggttatgcagaagaaaacgatgggttgggaagcgtccagcgagcgtatgtatccggaagatggcgcgttgaagggtgagatcaaacaacgtctgaagctgaaagacggcggccactatgatgcggaagtgaaaaccacttacaaggctaagaaaccggtgcagttgccgggtgcatacaatgttaacatcaagctggacattacctcccataacgaagattatacgatcgttgagcagtacgagcgtgccgagggtcgccacagcacgggcggtatggacgagctgtacaaataa

SEQ ID NO: 10 - protein, 236 aa, synthetic construct
  source 1..236: note = mCherry
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

(19) United States
(12) Patent Application Publication
(10) Pub. No.: US 2026/0228392 A1
(43) Pub. Date: Aug. 6, 2026

(54) DEEP LEARNING-BASED CODON OPTIMIZATION WITH LARGE-SCALE SYNONYMOUS VARIANT DATASETS ENABLES GENERALIZED TUNABLE PROTEIN EXPRESSION

(71) Applicant: ABSCI CORPORATION, Vancouver, WA (US)

(72) Inventors: Jahir Gutierrez Bugarin, Vancouver, WA (US); Joshua Meier, Vancouver, WA (US); Ariel Schwartz, Vancouver, WA (US); Miles Gander, Vancouver, WA (US)

(21) Appl. No.: 19/149,089

(22) PCT Filed: Jan. 12, 2024
(86) PCT No.: PCT/US2024/011311

§ 371 Date: Jul. 17, 2025

Related U.S. Application Data
(60) Provisional application No. 63/480,896, filed on Jan. 20, 2023.

(51) Int. Cl. G06F 30/27; G16B 25/00; G16B 40/20; G16B 40/30

(52) U.S. Cl. CPC G06F 30/27; G16B 25/00; G16B 40/20; G16B 40/30

(57) ABSTRACT

A method includes generating one or more DNA sequences using a machine learning model using coding sequences; comparing the DNA sequences to one or more natural DNA sequences; and determining a codon naturalness. A computing system includes a processor; and a memory having stored thereon computer-executable instructions that, when executed by the one or more processors, cause the computing system to: generate one or more DNA sequences using a machine learning model using coding sequences; compare the DNA sequences to one or more natural DNA sequences; and determine a codon naturalness. A non-transitory computer-readable medium has stored thereon instructions that, when executed by one or more processors, cause a computer to: generate one or more DNA sequences using a machine learning model using coding sequences; compare the DNA sequences to one or more natural DNA sequences; and determine a codon naturalness.

### INCORPORATION BY REFERENCE OF MATERIAL SUBMITTED ELECTRONICALLY

The Sequence Listing, which is a part of the present disclosure, is submitted concurrently with the specification as an XML file, named “58061P_SeqListing.xml,” which was created on Dec. 6, 2022 and is 15,285 bytes in size. The subject matter of the Sequence Listing is incorporated herein in its entirety by reference.

### FIELD OF THE DISCLOSURE

The present disclosure generally relates to a system and method for generalized codon optimization for increased protein expression via large-scale synonymous DNA variant datasets and deep learning, and more particularly, training and operating machine learning (ML) models to increase recombinant protein expression via dataset generation and model fine-tuning.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the disclosure.

Increasing recombinant protein expression is of broad interest in industrial biotechnology, synthetic biology and basic research. Codon optimization is an important step in heterologous gene expression that can have dramatic effects on expression level. Several codon optimization strategies have been developed to enhance expression, but these are largely based on bulk usage of highly frequent codons in the host genome, and can produce unreliable results.

Specifically, industrial production of recombinant proteins is a major component of biomanufacturing with a wide range of applications [1]. As of 2018, there are 316 licensed protein-based biopharmaceutical products with sales totaling at least $651 Billion since 2014 [2]. Microbial systems, such as E. coli [5, 6] have long been the workhorse of recombinant protein production, and are important biopharmaceutical manufacturing platforms with several advantages over conventional mammalian cells, such as scalability and affordability. As of 2022, there are at least 29 biopharmaceuticals produced in E. coli. Thus, increasing production efficiency by boosting protein expression in cellular hosts can have a significant impact on the affordability and availability of pharmaceuticals and other bio manufactured products [7].

The expression level of recombinant proteins depends on multiple factors, including the associated regulatory elements flanking the gene coding sequence (CDS) [8, 9], the culture conditions used for growing the production host cells [10], the metabolic state of such cells [11], or the co-expression of chaperones. Particularly, codon usage in the CDS is an important factor that has been exploited to increase recombinant protein expression in biotechnology and synthetic biology [12-15].

Codon usage patterns have been shown to affect expression via changes in translation rates, mRNA stability [27], protein folding and solubility [16,28]. Today, several commercial tools and algorithms exist to optimize codon usage in a CDS. These typically rely on sampling codons from the distribution of frequently observed codons in the host genome, maximizing the codon adaptation index (CAI) or codon-pair biases. However, these tools fail to account for long-range dependencies and complex codon usage patterns that arise in natural DNA sequences and do not reliably produce high expression yield CDs. For example, existing codon optimization tools may yield suboptimal DNA sequences that transcribe well in the host, but impede proper folding of the recombinant protein during translation. Alternatively, existing tools may yield sequences that enable proper folding but limit the stability and expression level of the transcribed mRNA, resulting in diminished yields of functional, soluble protein. For these reasons, developing novel codon optimization strategies capable of capturing these complex and long-range dependencies in DNA sequences is of high interest. Further, establishing language models that can predict DNA sequences and associated expression level for a given host will be a major step towards understanding the underlying principles governing gene expression level.

Natural language models based on Deep Learning (DL) have emerged as powerful tools for interrogating complex context-dependent patterns in biological sequences across application domains Although there are recent examples of DL-enabled codon optimization, these do not incorporate expression level information.

Thus, there is a need for methods, systems and platforms for improved techniques that incorporate expression-level information.

### BRIEF SUMMARY

In one aspect, a computer-implemented method for performing generalized codon optimization for improved protein expression includes (i) generating, via one or more processors, one or more DNA sequences using a machine learning model trained using coding sequences; (ii) comparing, via one or more processors, the DNA sequences to one or more natural DNA sequences to identify a subset of DNA sequences that correspond to a protein expression within a predefined range; and (iii) determining a codon naturalness for each of the subset of DNA sequences.

In another aspect, a computing system for performing generalized codon optimization for improved protein expression includes one or more processors; and one or more memories having stored thereon computer-executable instructions that, when executed by the one or more processors, cause the computing system to: (i) generate, via the one or more processors, one or more DNA sequences using a machine learning model trained using coding sequences; (ii) compare, via one or more processors, the DNA sequences to one or more natural DNA sequences to identify a subset of DNA sequences that correspond to a protein expression within a predefined range; and (iii) determine a codon naturalness for each of the subset of DNA sequences.

A non-transitory computer-readable medium has stored thereon instructions that, when executed by one or more processors, cause a computer to: (i) generate, via the one or more processors, one or more DNA sequences using a machine learning model trained using coding sequences; (ii) compare, via one or more processors, the DNA sequences to one or more natural DNA sequences to identify a subset of DNA sequences that correspond to a protein expression within a predefined range; and (iii) determine a codon naturalness for each of the subset of DNA sequences.

### BRIEF DESCRIPTION OF THE DRAWINGS

FIG. 1A depicts correlation of CAI of model generated sequences and natural counterparts, according to some aspects.

FIG. 1B depicts two representative % MinMax profiles of proteins within model training set, according to some aspects.

FIG. 1C depicts a density plot of cosine distances of % MinMax profiles in training set for model and randomly generated sequences, according to some aspects.

FIG. 1D depicts DNA sequence similarity for model and randomly generated sequences to natural sequence, according to some aspects.

FIG. 1E depicts a plot of normalized fluorescence expression values for GFP variants, according to some aspects.

FIG. 1F depicts a plot of fine-tuned expression level predictions on CO-T5 generated and commercial GFP Sequences, specifically, correlation of GFP model expression level predictions for GFP sequences from FIG. 1E and plate reader fluorescence measurements.

FIG. 1G depicts correlation of ALL model expression level predictions for GFP sequences from FIG. 1E and plate reader fluorescence measurements.

FIG. 2A depicts a schematic of the three tile synonymous mutant libraries constructed for GFP, according to some aspects.

FIG. 2B depicts density plots of normalized expression score for all three tile libraries, wherein expression scores show varied mutant score profiles based on tile position in CDS, and the plot indicates number of mutants measured within each tile, according to some aspects.

FIG. 2C depicts a correlation of GFP model predictions of a test set against measured expression scores, according to some aspects.

FIG. 2D depicts violin plots depicting model performance on out of distribution expression predictions for a holdout set consisting of the top 10% expressing variants (expression score measurements for the top 10% expressing holdout set and capped 90% training set are also shown), according to some aspects.

FIG. 2E depicts a density heatmap of expression scores between replicate sorts for the three GFP library tiles, wherein red lines indicate the parent GFP log-MFI, according to some aspects.

FIG. 2F depicts DNA nucleotide level Hamming distance from parent sequence for a first synonymous protein library, wherein the plot shows diverse DNA sequence space sampled within the expression library, according to some aspects.

FIG. 2G depicts DNA nucleotide level Hamming distance from parent sequence for a second synonymous protein library, wherein the plot shows diverse DNA sequence space sampled within the expression library, according to some aspects.

FIG. 2H depicts DNA nucleotide level Hamming distance from parent sequence for a third synonymous protein library, wherein the plot shows diverse DNA sequence space sampled within the expression library, according to some aspects.

FIG. 2I depicts a fluorescence distribution of GFP tile libraries, wherein debris, aggregates, and dead cells were excluded by parent gating prior to plotting the fluorescence signal for each library.

FIG. 2J depicts representative parent gating for GFP library sorts, wherein two singlets gates are drawn to exclude cellular aggregates regions previously identified by dual fluorescence of GFP and mCherry reporter strains.

FIG. 2K depicts parent gating for the VHH library was similar as described with respect to FIG. 4A, except PI was used to exclude non-permeabilized cells.

FIG. 3A depicts OD measurements of folA degenerate libraries after 24 hr of growth at increasing levels of TMP, wherein the plot depicts four replicate measurements and includes error bars are standard deviation, according to some aspects.

FIG. 3B depicts a Western Blot of DHFR at increasing levels of TMP for a folA degenerate library, wherein the blots show DHFR and GAPDH levels as a soluble protein level control and the barchart display depicts normalized band density values from the blots.

FIG. 3C depicts normalized expression score distribution from folA library selections, according to some aspects.

FIG. 3D depicts correlation of model predicted expression score values for folA variants and expression score measurements for a holdout test set, according to some aspects.

FIG. 3E depicts violin plots showing model performance of expression predictions for the top 10% expressing sequences in full dataset when trained on a capped 90% training set, wherein expression score measurements for the top 10% expressing holdout set and capped 90% training set are also shown, according to some aspects.

FIG. 3F depicts a heatmap of all-vs-all expression score Pearson R correlations between the four replicate screens from each of the six folA sub-libraries, wherein numbers in parentheses indicate number of variants in each library that passed all quality control filters, according to some aspects.

FIG. 3G depicts dose-response curves of synonymous folA codon variants from the top 1% (lavender), middle 2% (black), and bottom 5% (maroon) of score range from drug selection; and commercially optimized variants are shown in orange, wherein cultures were grown for 24 hours after expression induction in the presence of 1 μg/mL SMX and the indicated TMP concentration, according to some aspects.

FIG. 3H depicts area under the curve (AUC) of data in FIG. 3G from lines fit using GraphPad Prism, according to some aspects.

FIG. 3I depicts anti-DHFR Western blots on soluble protein fraction of cell lysates from folA strains shown in FIG. 3G and FIG. 3H, according to some aspects.

FIG. 3J depicts anti-DHFR Western blots on insoluble protein fraction of cell lysates from folA strains shown in FIG. 3G and FIG. 3H, according to some aspects.

FIG. 3K depicts quantification of data in FIG. 3I, normalized to signal from D3, according to some aspects.

FIG. 3L depicts quantification of data in FIG. 3J, according to some aspects.

FIG. 3M depicts correlation of soluble and insoluble DHFR protein expression with AUC data in FIG. 3H, normalized to signal from soluble fraction D3, according to some aspects.

FIG. 3N depicts a scatterplot showing log-MFI of control sequences when measured on Day 1 (tile 1) or Day 2 (tiles 2 and 3), wherein WT refers to the parental GFP sequence from which the libraries were derived, according to some aspects.

FIG. 3O depicts log-MFI expression score histograms for each tile before and after normalization, wherein the red vertical line indicates parent GFP Sequence log-MFI, according to some aspects.

FIG. 4A depicts a schematic of degenerate tiles applied to the anti-HER2 VHH parent sequence, according to some aspects.

FIG. 4B depicts normalized expression score distribution of VHH library variants, according to some aspects.

FIG. 4C depicts correlation of model predicted VHH expression scores and expression score measurements, according to some aspects.

FIG. 4D depicts violin plots depicting performance of model for a 90%-10% expression level training test split, according to some aspects.

FIG. 4E depicts Western blots of sampled strains from anti-HER2 VHH library, wherein blots show levels of VHH molecule and GAPDH solubility control, according to some aspects.

FIG. 4F depicts levels of soluble protein as measured by band density of Western Blots from FIG. 4E, wherein Levels are calculated as density of VHH/GAPDH, then normalized to the IDT parent strain levels, according to some aspects.

FIG. 4G depicts correlation of strains from Western Blot in FIG. 4E with ACE derived expression scores for strains, according to some aspects.

FIG. 4H depicts a density heatmap of expression scores between replicate sorts for the VHH library, according to some aspects.

FIG. 5A depicts model-designed protein sequences compared against baselines, specifically FU/OD600 values measured by plate reader for mCherry sequences designed by the ALL model to maximize or minimize expression, compared to various optimization baselines, wherein values are an average of two replicate measurements.

FIG. 5B depicts a barchart showing fraction of designs in upper quartile of all measured mCherry variants for each sequence group, excluding the ALL (bottom) set.

FIG. 5C depicts ACE functional expression measurements, mean fluorescent intensity (MFI), for anti-SARS-COV-2 VHH sequences designed by the model to maximize or minimize expression, compared to various optimization baselines, wherein expression values are average of two replicate measurements.

FIG. 5D depicts a barchart showing fraction of designs in the upper quartile of all measured anti-SARS-COV-2 VHH variants for each sequence group, excluding the ALL (bottom) set.

FIG. 6A depicts plate reader RFU/OD600 values from 24 variants from the three GFP tile libraries, according to some aspects.

FIG. 6B depicts fluorescent cytometry readings of the 24 variants from each tile, according to some aspects, wherein error bars are standard deviation of recorded events, according to some aspects.

FIG. 6C depicts correlations of Plate Reader and Cytometer fluorescent measurements, according to some aspects.

FIG. 6D depicts a table of sequencing characterization of 24 colonies from each tile, including minimal levels of non-GFP coding variants in sampled variants, according to some aspects.

FIG. 6E depicts soluble protein levels as measured by Western Blot of a subset of variants per tile, wherein values are reported as a percentage of parent GFP sequence levels, according to some aspects.

FIG. 6F depicts insoluble protein levels of variants from panel E as measured by Western Blot, wherein values are reported as a percentage of parent sequence soluble protein level, according to some aspects.

FIG. 6G depicts plate Reader fluorescent values correlated with soluble protein levels as measure by western blot, wherein the plot shows variants from each tile appearing in panel FIG. 6E, according to some aspects.

FIG. 6H depicts correlation of Western Blot soluble protein to geometric mean fluorescence of variants from each tile, according to some aspects.

FIG. 7 depicts soluble protein level of GFP variants measured via western blot vs Normalized Expression Score, according to some aspects.

FIG. 8A depicts codon importance in predicting anti-HER2 VHH expression values in XGBoost model trained on one-hot encoded representations, wherein the chart shows the top 20 most important features (codons) used by the XGBoost model to predict expression values; and each codon is numbered according to its position in the sequence and the number in brackets denotes its positional quartile, where 1 means the codon is found in the first fourth of the sequence and a 4 means the last fourth of the sequence.

FIG. 9A depicts a distribution of randomly sampled and scored sequences for design of mCherry DNA sequences, wherein the distribution is of a representative subset of sampled and scored sequences using the GFP full model.

FIG. 9B depicts distribution of a representative subset of sampled and scored sequences using the ALL model.

FIG. 9C depicts a distribution of randomly sampled and scored sequences for design of SARS-COV-2 VHH DNA sequences, specifically distribution of a representative subset of sampled and scored sequences using the VHH model.

FIG. 9D depicts a distribution of a representative subset of sampled and scored sequences using the ALL model.

FIG. 10A depicts mCherry and anti-SARS-COV-2 VHH model and commercial designs full data set, specifically, a boxplot of all model and commercially designed sequences of mCherry, including the high expression level of the GFP (bottom) 10 designs.

FIG. 10B depicts a Boxplot of all model and commercially designed sequences of anti-SARS-COV-2 VHH.

FIG. 11A depicts correlation of ALL model scores from mCherry and anti-SARS-COV-2 VHH variants tested in FIG. 5A-FIG. 5D, specifically, correlation plot of ALL model scores and functional expression fluorescence measurements of mCherry sequences from FIG. 5A

FIG. 11B depicts a correlation plot of ALL model scores and ACE functional expression measurements of SARS-COV-2 VHH sequences from FIG. 5C.

FIG. 12 depicts an exemplary computer-implemented method, according to some aspects.

FIG. 13 depicts an exemplary computing environment, according to some aspects.

### DETAILED DESCRIPTION

### Overview

Although the following text sets forth a detailed description of numerous different embodiments, it should be understood that the legal scope of the description is defined by the words of the claims set forth at the end of this text. The detailed description is to be construed as exemplary only and does not describe every possible embodiment since describing every possible embodiment would be impractical, if not impossible. Numerous alternative embodiments could be implemented, using either current technology or technology developed after the filing date of this patent, which would still fall within the scope of the claims.

It should also be understood that, unless a term is expressly defined in this patent using the sentence “As used herein, the term” “is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. § 112 (f).

The present techniques include machine learning (ML) techniques (e.g., deep contextual language models) that may learn the codon usage rules from natural protein coding sequences (e.g., across members of the Enterobacterales order). The present techniques may include, next, fine-tuning these models using many (e.g., 150,000 or more) functional expression measurements of synonymous coding sequences from a number (e.g., three) of proteins to predict expression in E. coli from codon usage.

The present techniques demonstrate that these such ML models are capable of recapitulating natural context-specific patterns of codon usage, and accurately predicting expression levels across synonymous sequences. Further, the present techniques demonstrate that expression predictions may generalize across proteins unseen during training, allowing for the in silico design of gene sequences for optimal expression. As will be appreciated by those of ordinary skill in the art, the present techniques provides novel and reliable techniques for tuning gene expression with many potential applications in biotechnology and biomanufacturing.

As noted above, recent examples of DL-enabled codon optimization do not incorporate expression level information. The present techniques improve upon conventional systems by showing that DL models traditionally used for NLP tasks are able to learn meaningful patterns of codon usage and generate sequences mimicking natural codon usage profiles when trained on genome-scale CDS data. In particular, the present techniques demonstrate that language models are able to learn from long-range patterns of codon usage and generate sequences mimicking natural codon usage profiles when trained on genome-scale CDS sequence data. Further, the present techniques demonstrate that optimizing gene sequences for natural codon usage patterns alone may not guarantee high protein expression level; rather, additional optimization based on sequence to functional expression level data may be necessary to reliably identify gene sequences with high expression.

The present techniques extend the use of language models for codon optimization by predicting protein expression levels via training on a large collection of paired sequence to expression pairs for three distinct recombinant proteins. These functional soluble protein expression datasets were generated via multiple assays including the described Activity-specific Cell Enrichment (ACE) assay, a Sort-seq method and antibiotic selection. Taken together, the full dataset accounts for 154,166 total functional expression measurements of synonymous full gene sequences and likely represents the largest dataset of its kind. With this dataset, the trained models predict expression level of unseen sequence variants with high accuracy. Finally, the present techniques predict and experimentally validate high-expressing sequences for two proteins outside the model training set, demonstrating the generalized ability for the model to design DNA sequences for optimal expression of functional proteins.

The present techniques may include validation experiments demonstrating that gene sequences with natural codon usage profiles express comparably to gene sequences generated with commercial codon optimizers but do not necessarily express at high levels. Further, in response to these validation experiments, the present techniques may improve upon conventional techniques by predicting protein expression levels via ML-based training (e.g., using a quantitative functional dataset, across three different proteins).

In some aspects, functional expression data may be generated via Activity-specific Cell Enrichment (ACE) assay [31, 36], a Sort-seq method and/or antibiotic selection.

For example, in some aspects, a training dataset consists of a large number (e.g., at least 5000, at least 10000, at least 25000, at least 50000, at least 100000, at least 150000, etc.) of total expression measurements of synonymous full gene sequences. This dataset may be used to study and model CDS-dependent protein expression, in some aspects. This dataset may be used to train one or more ML models to predict an expression level(s) of unseen sequence variants with high accuracy in the context of a single protein.

Further, in some aspects, multi-task learning may be used across multiple proteins to increases performance. In some aspects, the present techniques may include design and testing of high-expressing sequences of proteins outside of model training sets. Advantageously, the present techniques demonstrate models capable of designing DNA sequences for optimal expression of soluble, functional proteins.

Optimizing synonymous DNA sequence for protein expression is a significant challenge with a vast amount of potential solutions. For example, a 100 amino acid protein has roughly 10⁴⁷ possible permutations. While optimization strategies exist based on codon usage [16], codon pair bias and presence or absence of sequence motifs [12, 14] these approaches lack the ability to capture complex long-range patterns across sequences in this extremely diverse solution space. Additionally, current optimization strategies can yield unreliable results.

The present techniques represent a significant improvement over conventional techniques, by methods and systems for deep contextual language models capable of capturing natural codon usage patterns and predicting expression level of proteins based on DNA sequence.

While training models on genomic sequence allows for the generation of sequences with natural attributes, the state of the art for DL-enable codon optimization, this alone is not sufficient to consistently generate synonymous DNA sequences with high expression levels. To overcome this challenge in conventional techniques, the present techniques include generating a large functional protein expression dataset across a plurality (e.g., three) of individual proteins and fine-tuned models for expression level predictions. This dataset is the largest of its kind and will serve as a resource for future efforts to model protein expression based on DNA coding sequence. The present techniques show these models can be applied to design coding sequences with specified expression levels.

Codon optimization for protein expression is a significant challenge at least due to (1) the large number of possible codon permutations and (2) the complex downstream effects that codons have on processes like transcription, translation, protein folding, and function. For example, a peptide with 100 amino acid protein has 1.63107 possible synonymous DNA sequences. While optimization strategies exist based on codon usage, codon pair bias, and presence or absence of sequence motifs, these approaches lack the ability to capture complex long-range patterns across sequences associated with expression level in this extremely diverse space. Here the present techniques demonstrate the ability of deep contextual language models to capture natural codon usage patterns and predict expression level of proteins based on DNA sequence.

The present techniques show that while training models on genomic sequence from a given taxon allows for the generation of sequences with natural attributes, the state of the art for DL-enable codon optimization, this alone is not sufficient to consistently generate synonymous DNA sequences with high protein expression levels. To overcome this challenge, the present techniques generated the largest ever functional protein expression dataset across three individual proteins and fine-tuned models for protein expression level predictions. The present techniques show these models can predict CDSs that produce proteins at specified expression levels in the context of a single protein. The present techniques also show the model's ability to accurately rank sequences with protein expression levels higher than observed in the training set, which can save time and resources by prioritizing predicted high yield variants for in vivo testing. Additionally, we show that training on the functional expression dataset imparts models with the ability to predict expression of DNA variants for proteins outside the training set. Further, the present techniques demonstrate that this generalizability can be harnessed to design DNA sequence variants with specified expression levels for new proteins. The model guided design techniques discussed herein outperform benchmark tools for optimizing and tuning protein expression.

It is expected that models may be extended by increasing the number of diverse protein sequences in training data. The results discussed herein indicate that new training examples can increase accuracy of predictions, which may further improve the generalized model. An additional area of interest would be extending the present techniques to other organisms beyond E. coli.

The present techniques improve biological outcomes at the DNA level, including improving transcription of a DNA coding sequence, improving mRNA stability, improving translation rates, improving protein expression, improving protein stability, improving proper folding, improving protein stability, improving protein function, improving protein binding, improving protein activity, etc. Thus, the present disclosure provides a computer-implemented method to improve transcription of a DNA coding sequence, mRNA stability, translation rates, protein expression, protein stability, proper folding, protein stability, protein function, protein binding, protein activity, etc. comprising the steps of generating, via one or more processors, one or more DNA sequences using a machine learning model trained using coding sequences; comparing, via one or more processors, the DNA sequences to one or more natural DNA sequences to identify a subset of DNA sequences that are associated with high expression; and determining a codon naturalness for each of the subset of DNA sequences.

Finally, the present techniques focus on optimizing protein expression through codon usage, but conventional techniques apply DL or traditional ML to alternative regulatory elements such as promoters, ribosome binding sites and terminators. In principle, models accounting for multiple elements involved in expression regulation could be combined to generate a unified model for protein expression.

Taken together the present techniques represent a generalized, effective and efficient method for optimizing expression of recombinant proteins via codon choice superior to existing techniques. The models discussed herein for tuning protein expression improve biomanufacturing and biopharmaceutical availability. The present techniques demonstrate the value of applying DL to complex biological sequence space, while providing a framework for increasing protein yield in biological systems.

### Exemplary Transformer Nlp Models for Predicting Natural-Like DNA Sequences

### Generative Deep Language Models Optimize CDSs for Natural Sequence Attributes But May not Reliably Create High Expressing Variants

The present techniques demonstrate, inter alia, that generative deep language models optimize CDSs for natural sequence attributes but may not not reliably create high expressing variants.

The present techniques include the use of language models for codon optimization by predicting protein expression levels via training on a large collection of sequence-expression pairs for three distinct recombinant proteins. These functional soluble protein expression datasets were generated via multiple assays including the previously described Activity-specific Cell Enrichment (ACE) assay, a Sort-seq method and antibiotic selection. Taken together, the full dataset accounts for 154,166 total functional expression measurements of synonymous full gene sequences and represents the largest dataset of its kind. The full dataset serves as a resource for further study and modeling of CDS-dependent protein expression in the field. With this dataset, the present trained models predict expression level of unseen sequence variants with high accuracy. The present techniques may further include design and test of high-expressing sequences of proteins outside the model training set, demonstrating the generalized ability for the model to design DNA sequences for optimal expression of soluble, functional proteins.

One common approach for codon optimization is a text translation task, where a sentence written in the language of protein, amino acids, is translated to a sentence in DNA nucleotides, conditioned on the identity of the host organism to maximize the natural profile of the CDS. The present techniques may include repurposing the T5 language model architecture by training on a dataset, named Protein2DNA, consisting of all protein coding and protein-CDS pairs from high quality genomes of the Enterobacterales order (taxonomic identifier 91347). It should be appreciated that other datasets may be used, and that the dataset may relate to any organism.

Specifically, the present techniques may include training one or more models, referred to as CO-T5, on the single task of protein-to-DNA translation, conditioned on the taxonomy of the host genome. Training in this way allows the model to learn rich, shared gene sequence patterns across organisms. By providing the identity of the host organism, CO-T5 learns to output CDSs specific to each taxonomy. The present models represent the first deep generative language model for full length CDSs. To test CO-T5's ability to learn natural sequence patterns, the present techniques may include generating DNA sequences from a holdout set of proteins from various members of the Enterobacterales order, and comparing the generated sequences to the endogenous versions.

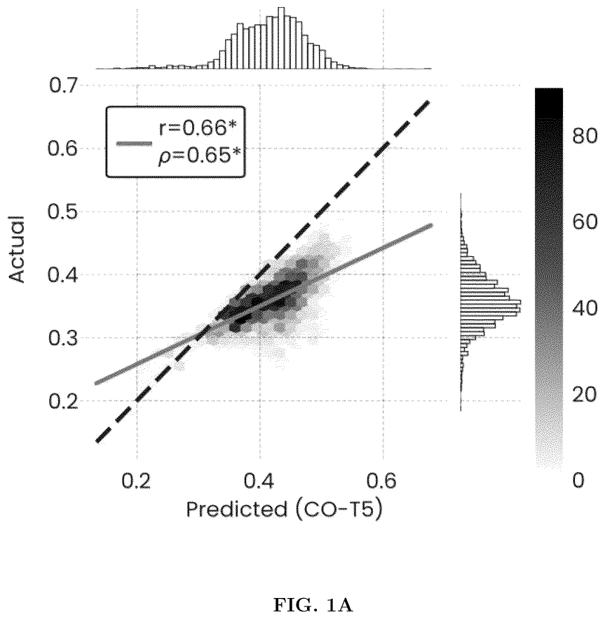

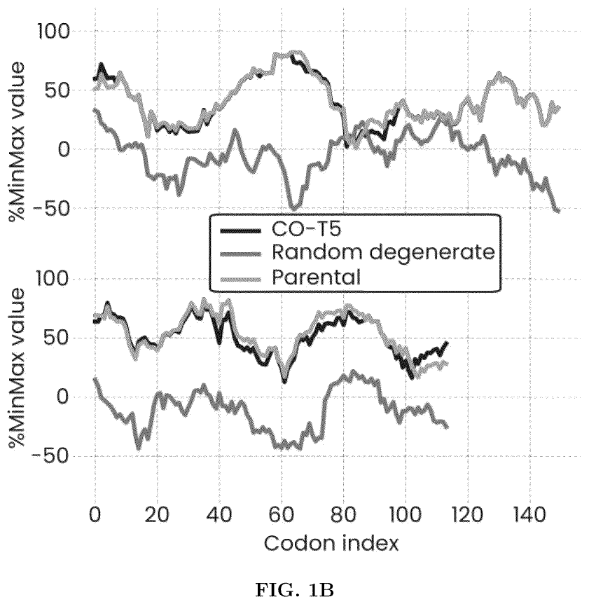

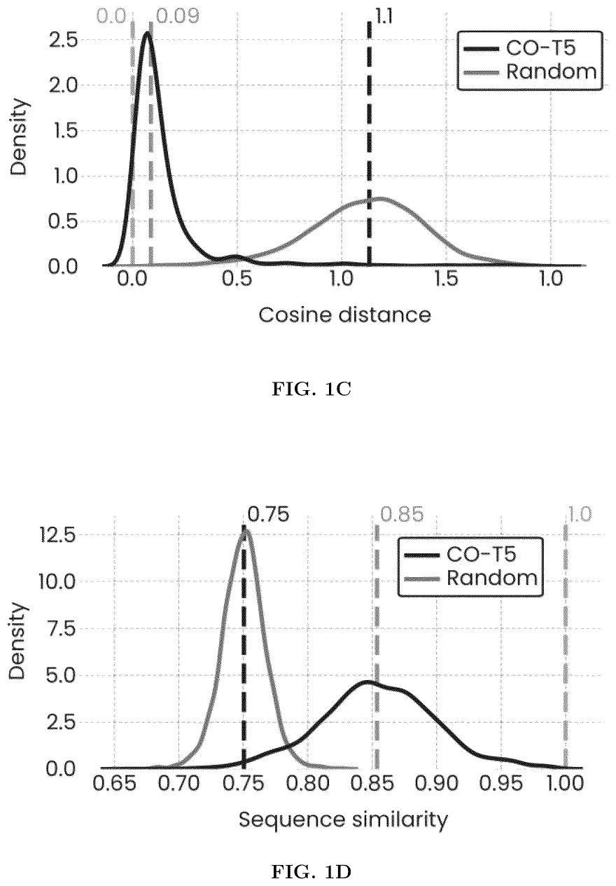

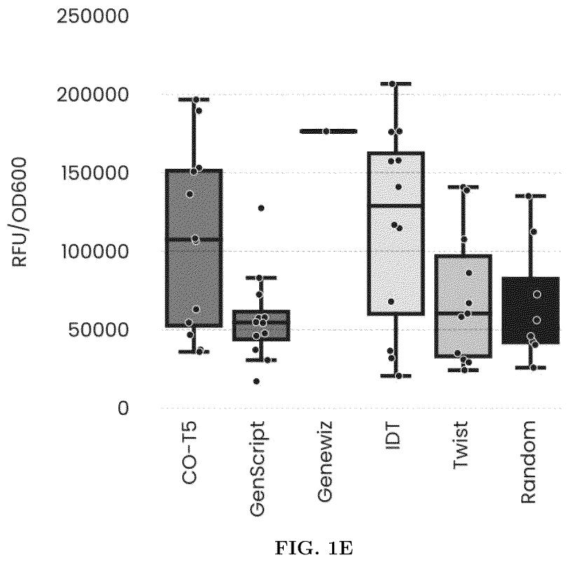

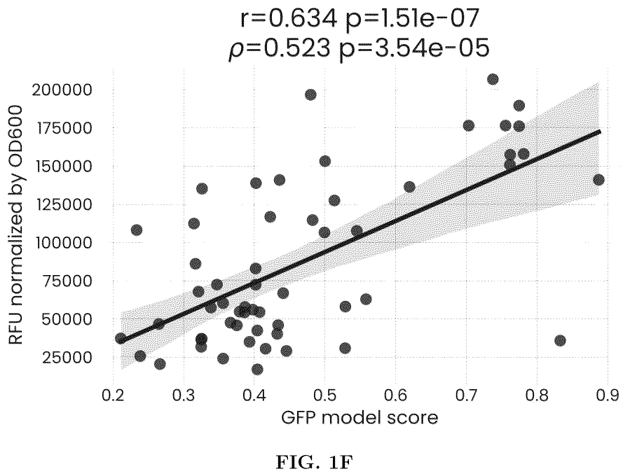

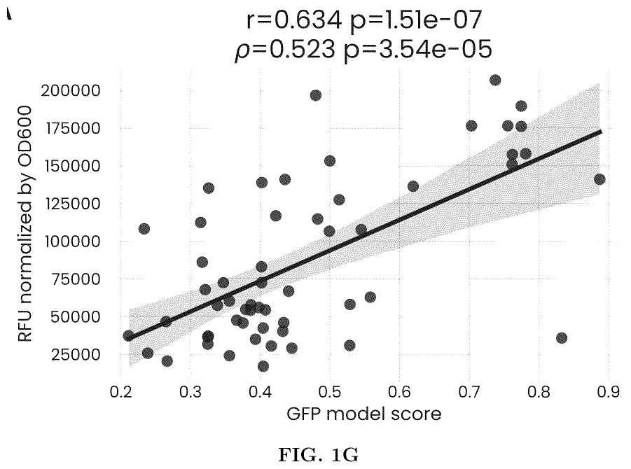

As shown in FIG. 1A, generated sequences and their natural counterparts have similar Codon Adaptation Index (CAI)], a measure of host codon frequency in a CDS. To determine if model-generated sequences have natural long-range codon usage patterns the present techniques may also include investigating the % MinMax profiles across generated sequences compared to their natural counterparts. % MinMax measures the prevalence of rare codons in a sliding window, where lower values indicate clusters of rare codons. For comparison, the present techniques may include generating synonymous sequences for each natural amino acid sequence by randomly sampling degenerate codons at each position. Empirical testing demonstrated that the % MinMax profiles of CO-T5-generated sequences are remarkably similar to their natural counterparts (FIG. 1B, FIG. 1C) compared to random degenerate sequences, demonstrating the ability of the model to recapitulate DNA sequences with natural attributes. Empirical testing wherein sequence similarities (FIG. 1D) were computed found that CO-T5-generated sequences, from holdout examples, have an average sequence similarity of 85% to their natural counterparts in contrast to an average of 75% for random synonymous sequences.

### Exemplary Protein2DNA Dataset Construction

The present techniques may include computer-executable instructions that construct a Protein2DNA data set used to train the above-referenced NLP model by applying several selection criteria. The instructions may include downloading protein and DNA records from a public database (e.g., RefSeq) and/or taxonomic identifiers from a public database (e.g., the NCBI Taxonomy database). In some aspects, all taxonomic kingdoms are considered when downloading these records.

The instructions may include filtering the genomic records according to metadata included in the records. Specifically, the present techniques may include excluding genomes that have a status of other than “reference genome” or “representative genome,” in some aspects. The instructions may further include filtering out (i.e., excluding) incomplete genomes by using a “Complete Genome” or “Full” tag in the record metadata.

The instructions may include scanning each downloaded record for coordinate align-ment between the DNA and its amino acid translation, dropping (i.e., excluding) those records with inconsistent protein-DNA mapping. For genes, in some aspects, records labeled as “pseudo” may be excluded and only those with a “cds” or “translation feature” tag included in the constructed dataset.

The instructions may include keeping only records with a valid stop codon were included (i.e., excluding records with invalid stop codon). Further, the instructions may include checking, for each corresponding protein sequence, that the sequence was not truncated (i.e., that the protein sequence starts with an “M” and ends with a “*” or stop symbol). Finally, the instructions may include discarding any sequences lacking canonical DNA bases or amino acids.

### Processing of Protein2DNA Dataset Entries

In order to train the language models, the present techniques created a dictionary mapping relevant characters or words from the Protein2DNA dataset to unique tokens. Briefly, the present techniques assigned unique tokens to (1) each of the 20 amino acids as well as the stop symbol; (2) each of the 64 codons; (3) each of the taxonomic ranks (Kingdom, Phylum, Class, Order, Family, Genus, Species, Strain, and Genetic Code); and (4) each of the 10 numeric digits (0-9) to represent taxonomic identifiers. In this way, each entry of the Protein2DNA dataset is converted to a language model-compatible representation by tokenizing each of the words comprising its taxonomy, amino acid sequence, and DNA sequence. Specifically, the order of the tokenized words for a Protein2DNA entry whose corresponding CDS has N codons is:
[<Kingdom> <Kingdom number> <Phylum> <Phylum number> <Class><Class number> <Order> <Order number> <Family> <Family number> <Genus><Genus number> <Species > <Species number> <Strain > <Strain number> <1st amino acid> <2nd amino acid> . . . < (N-1)th amino acid > <Stop symbol>]
for the input text, and
[<1st codon> <2nd codon >. <Nth codon >]
for the label.

### Training of CO-T5 Model

The present CO-T5 architecture may be based on the T5ForConditionalGeneration model and its PyTorch implementation within the HuggingFace framework. In some aspects, other implementations may be used, including those written from scratch. The model may include, for example, 12 attention layers, the hidden layer size may be 768 and the intermediate layer size may be 1152. In some aspects, different model layers and layer parameters may be used. Model training may be performed in a supervised manner, whereby the model is shown a tokenized input text (taxonomy+amino acid sequence) and the corresponding tokenized label text (CDS sequence) as described above. The present techniques may use a learning rate (e.g., of 4×10⁻⁴ with 1000 warm-up steps) and no weight decay, in some aspects. The final CO-T5 model may be trained for a number (e.g., 84) of epochs, which may correspond to the point where both the training and validation loss converge while avoiding overfitting. The computing system depicted in FIG. 13 may be used to perform the training and operation of the present models.

### Training of CO-BERTa Model

In some aspects, the present CO-BERTa architecture may be based on the RobertaFor-MaskedLM model and its PyTorch implementation within the HuggingFace framework. In some aspects, other implementations may be used, including those written from scratch. The model may include, for example, 12 attention heads and 16 hidden layers. The hidden layer size may be 768 and the intermediate layer size 3072. Model training may be performed in a self-supervised manner following a dynamic masking procedure with a special <MASK> token. For masking, the present techniques may use the DataCollatorForLanguageModeling class from the Hugging Face framework, for example, to randomly mask codon tokens with a probability of 20%. Entries from the Protein2DNA datasets may be processed in the same way as for the CO-T5 model described above, in some aspects. Training may be performed with the LAMB optimizer with learning rate of 10⁻⁵, linear rate decay with weight of 0.01, 1000 steps of warm-up, a gradient clamp value of 1, and dropout probability of 20%. The model may be trained for a number (e.g., 100) of epochs, corresponding to the point where both the training and validation loss converge while avoiding overfitting. The computing system depicted in FIG. 13 may be used to carry out the training and operation of the present models.

### Finetuning of CO-BERTa Model

The present techniques may fine-tuned the pre-trained CO-BERTa model by adding a dense hidden layer with for example, 768 nodes followed by a projection layer with a single output neuron (regressor). All layers may remained unfrozen to update all model parameters during training. Training may be performed with the AdamW optimizer, with a learning rate of, for example, 10⁻⁵, a weight decay of 0.01, a dropout probability of 0.2, a linear learning rate decay with 1000 warm up steps, and mean-squared error (MSE) as the loss function. Of course, other training parameters may be selected, in some instances.

### Codon Naturalness Score

The concept of antibody Naturalness score was previously introduced and shown to denote how natural a given antibody sequence is according to a pre-trained language model. The present techniques introduce the concept of a codon Naturalness score, which is a model-generated score of how natural a codon sequences is in the context of the host genome. Codon Naturalness score may be considered analogous to the concept of antibody Naturalness score with respect to antibodies. Formally, the codon Naturalness score of a CDS may be defined as the inverse of the pseudo-complexity value computed by the CO-T5 language model.

The present techniques may include testing whether the natural-like generated DNA sequences from the model were associated with high expression, and comparing them to other commonly used optimization algorithms. Empirical testing showed the present techniques expressed sequences generated by the CO-T5 model with high naturalness, sequences designed with commercially available codon optimizers, and random sequences, as shown in FIG. 1E.

While the CO-T5-generated sequences were among the highest expressors, empirical testing has shown no statistically significant difference was found by one-way ANOVA between CO-T5 and commercial algorithms, except GenScript. Showing that highly natural CDSs do not necessarily express highly, likely due to natural expression variation across genes. Based on these findings, the present techniques next attempted to use supervised learning and fine-tuning to create language models that can accurately associate specific codon sequences with expression values, with the aim of identifying CDSs with high expression levels.

The present techniques may include fine-tuning expression level predictions on T5 generated and commercial GFP Sequences. FIG. 1F depicts correlation of GFP model expression level predictions for GFP sequences from FIG. 1E and plate reader fluorescent measurements.

FIG. 1G depicts correlation of ALL model expression level predictions (see Table 145) for GFP sequences from FIG. 1E and plate reader fluorescent measurements.

### Exemplary Synonymous Mutant Functional Protein Expression Datasets

### Exemplary Masked-Language Models

The present techniques may include masked-language models to learn to map CDS to expression levels across a synonymous mutant dataset, in some aspects.

Given the varied expression levels of CO-T5-model generated high Naturalness score sequences the present techniques devised a supervised learning approach to map specific codon sequences to expression values. To build models that can learn this mapping, the present techniques first pre-trained a masked language model called CO-BERTa using the ROBERTa architecture on the same Enterobacterales dataset used for CO-T5. The present techniques then collected three large-scale datasets of functional expression values for synonymous codon sequences using three different recombinant proteins.

These data were used to fine-tune CO-BERTa for the task of predicting expression from a given CDS. As many applications necessitate properly folded soluble proteins, the present techniques focused on measuring functional protein levels rather than total protein, although functional assays can often be more difficult to develop.

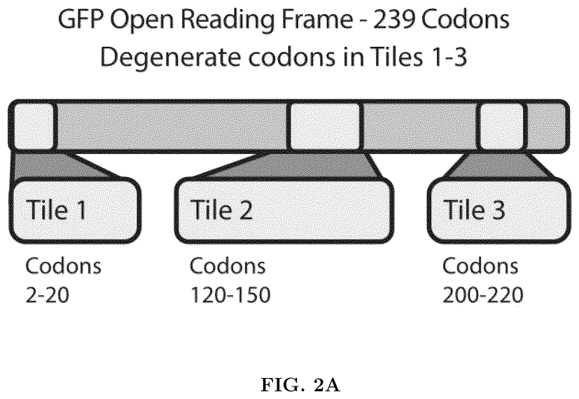

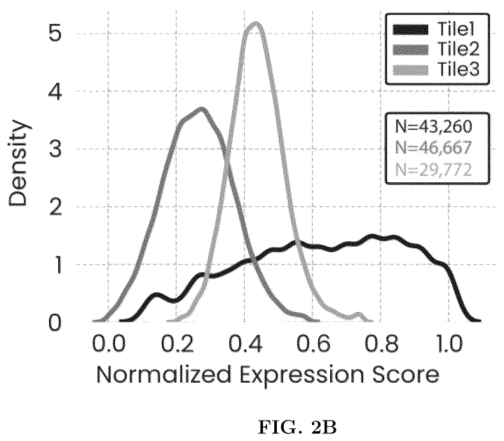

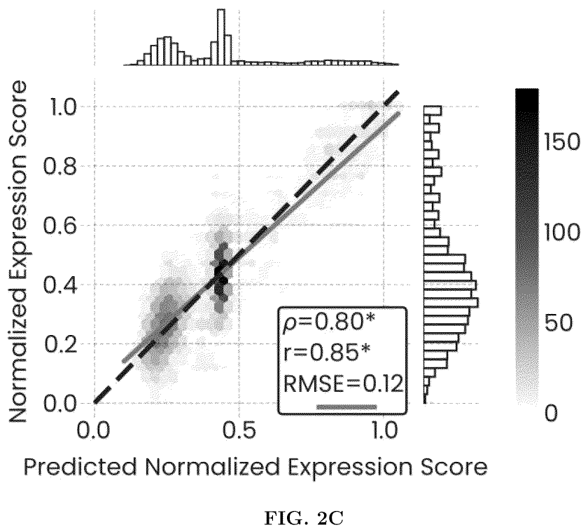

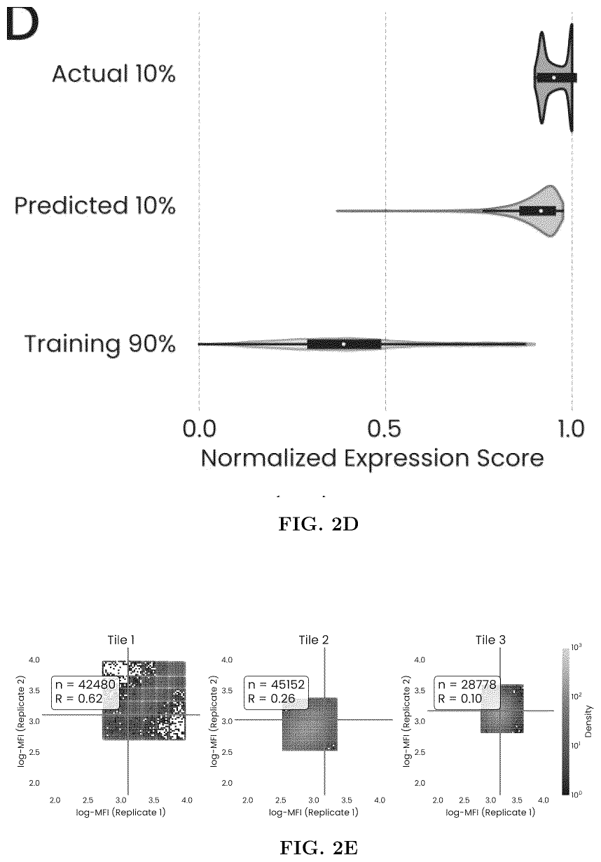

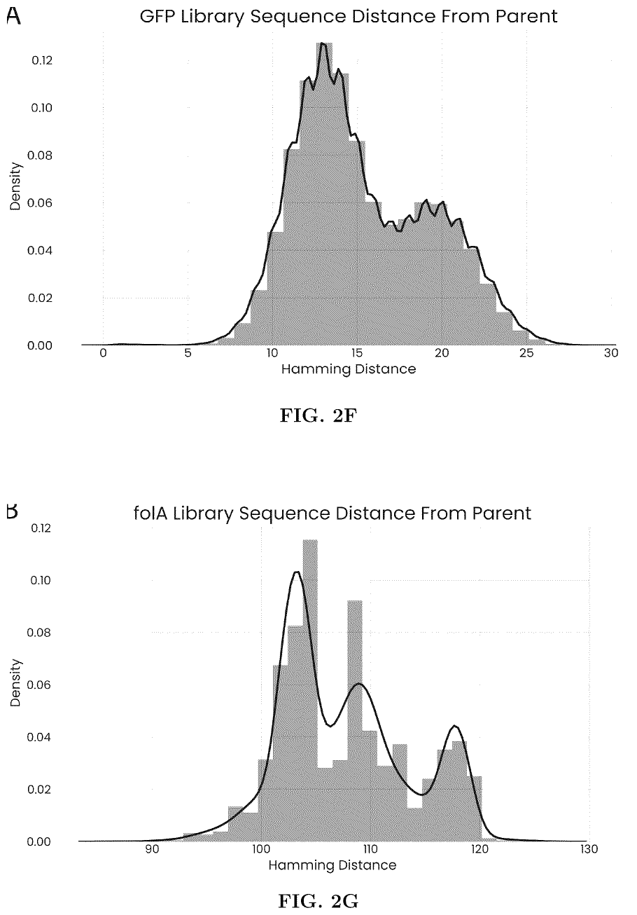

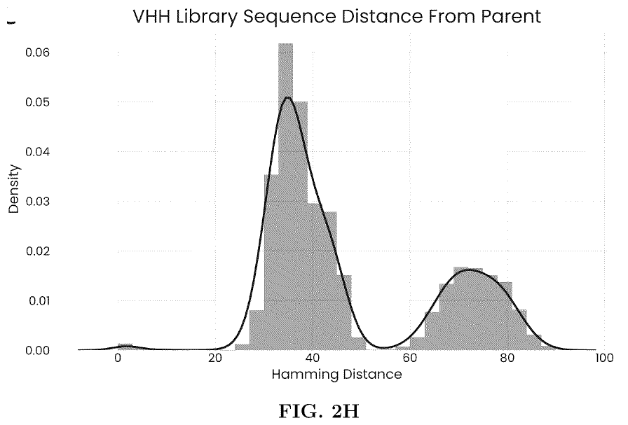

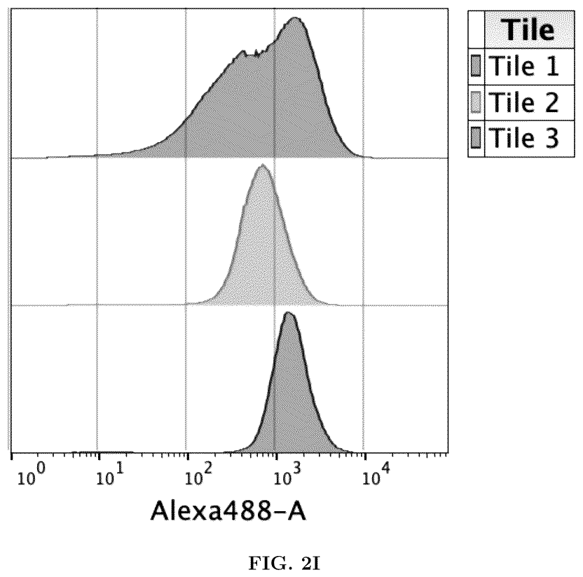

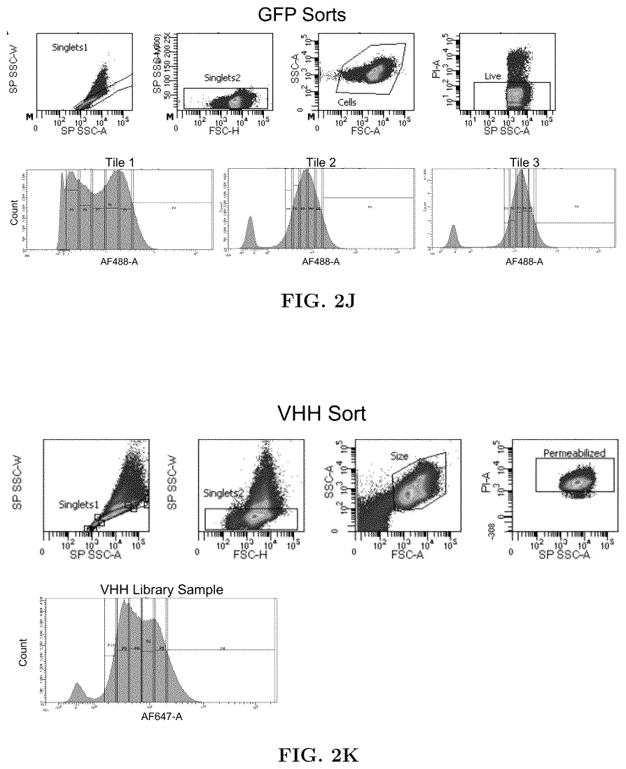

The present techniques used Green Fluorescent Protein (GFP) as a first protein to generate synonymous functional mutant expression data. GFP, a 238 amino acid protein has 2.12110 possible coding variants, many more than is feasible to measure in the laboratory. To focus the search of this massive synonymous mutational space, for effective quantitative screening, the present techniques selected three regions, or tiles, along the CDS known to affect protein expression (FIG. 2A). For each tile, the present techniques constructed a library of degenerate synonymous sequences starting from a parental GFP CDS (Table 1). The present techniques then cloned each tile as an independent library of synonymous GFP sequences. This resulted in a highly diverse set of GFP sequence libraries in which only one tile is modified in the CDS at a time (FIG. 2F). The present techniques then used a Sort-seq method to measure the expression of synonymous GFP sequence mutants in the three libraries (FIG. 2I, FIG. 2J, FIG. 2K and FIG. 2E).

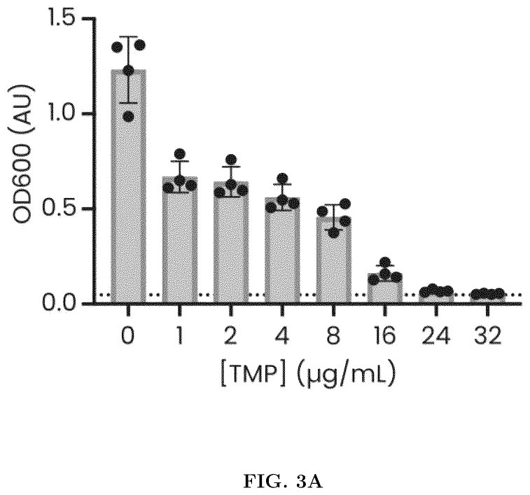

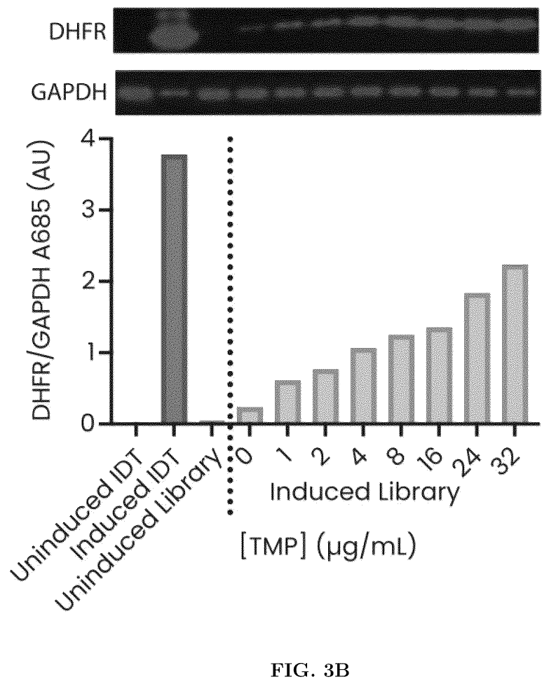

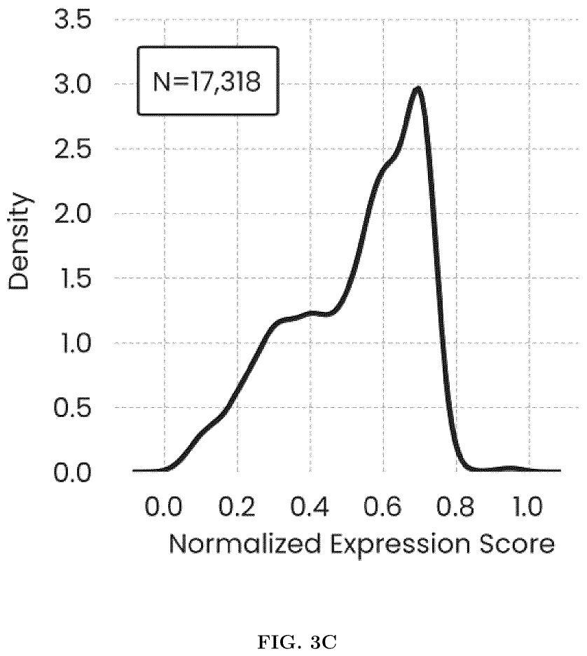

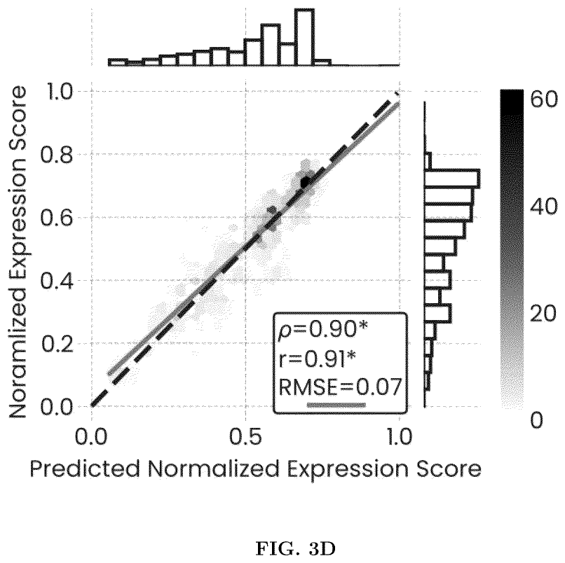

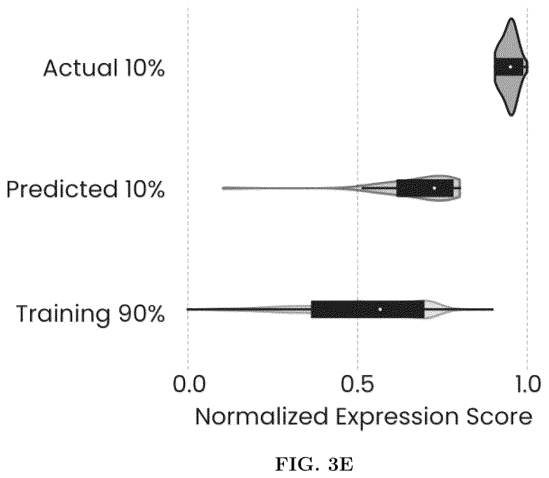

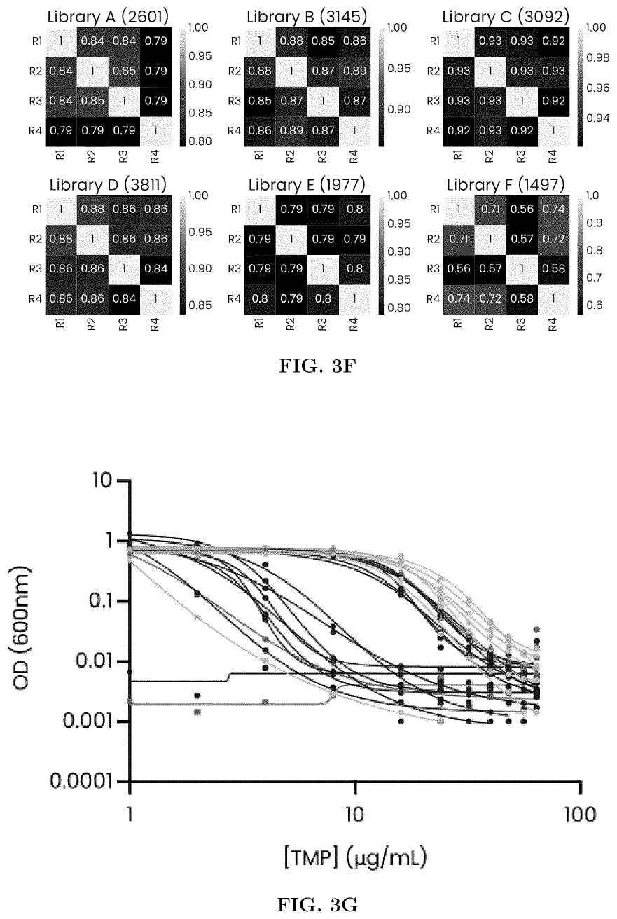

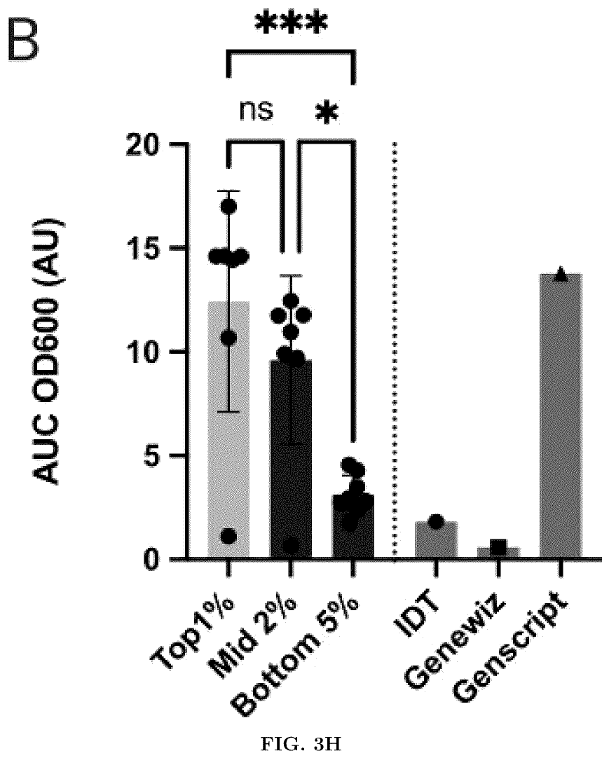

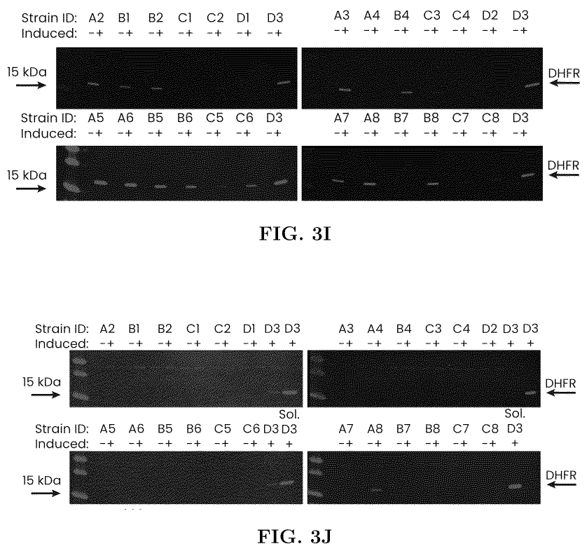

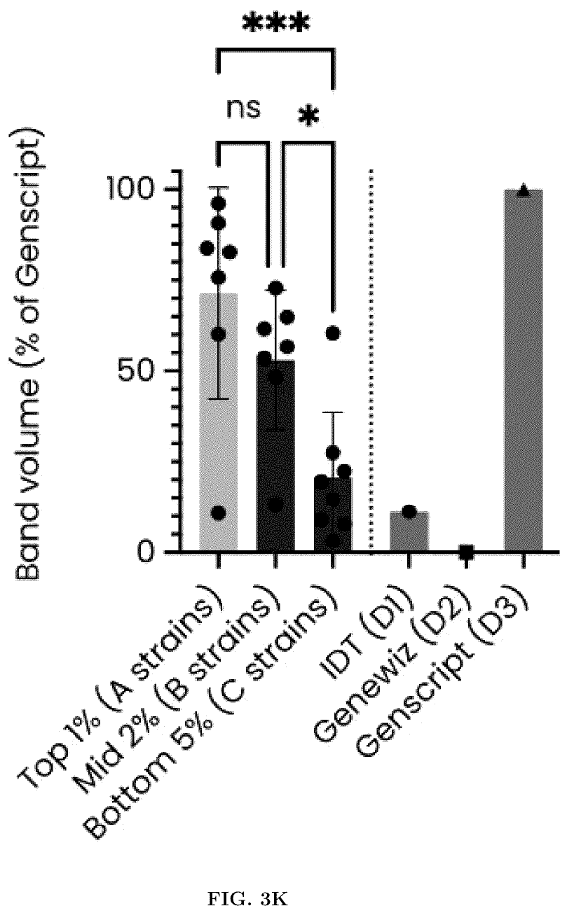

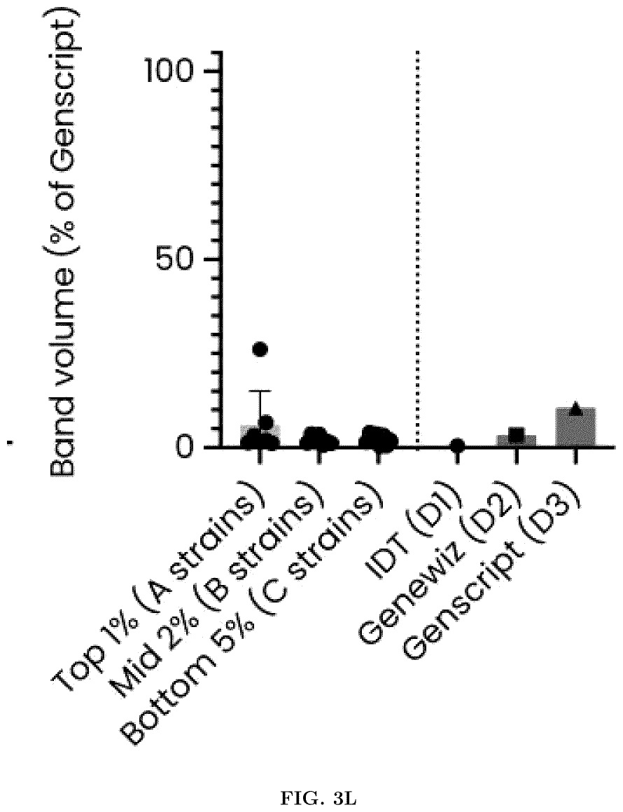

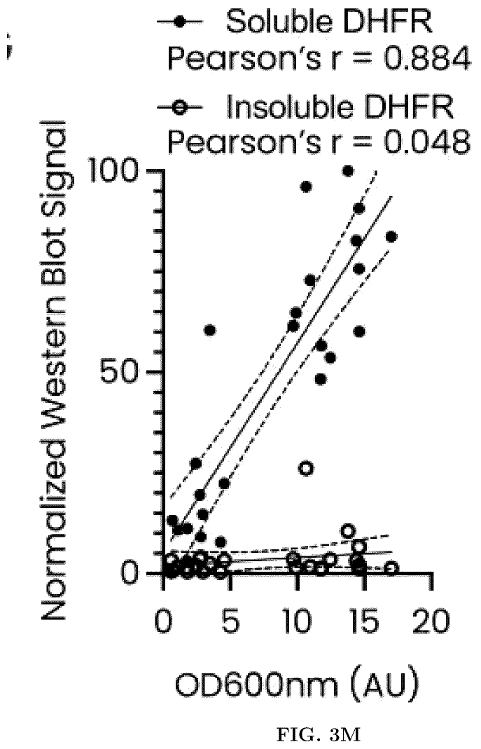

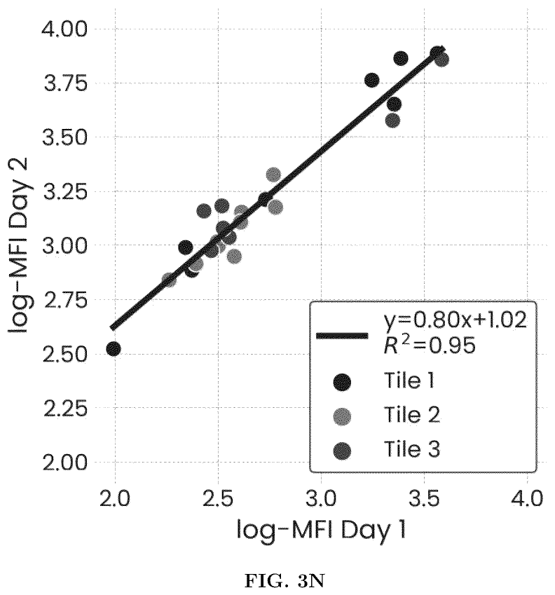

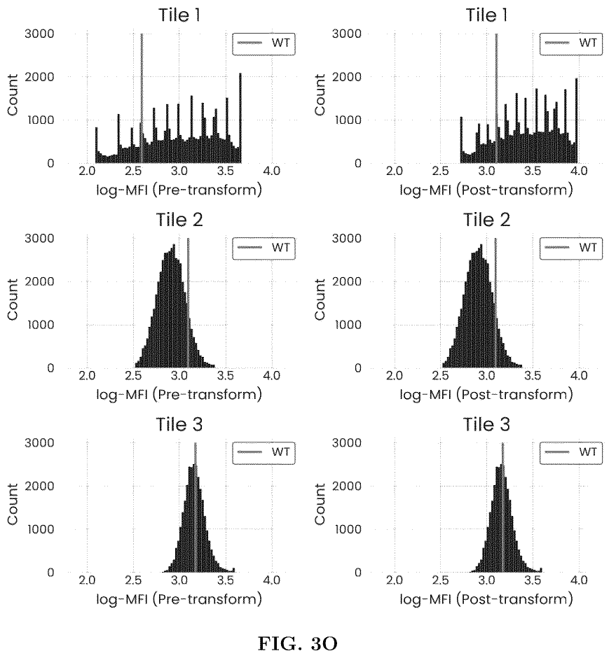

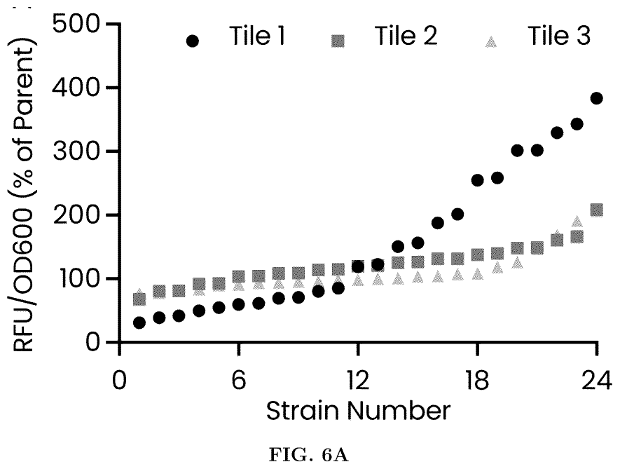

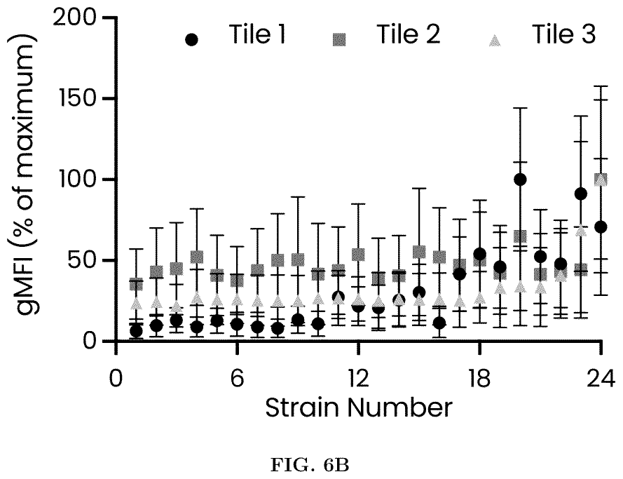

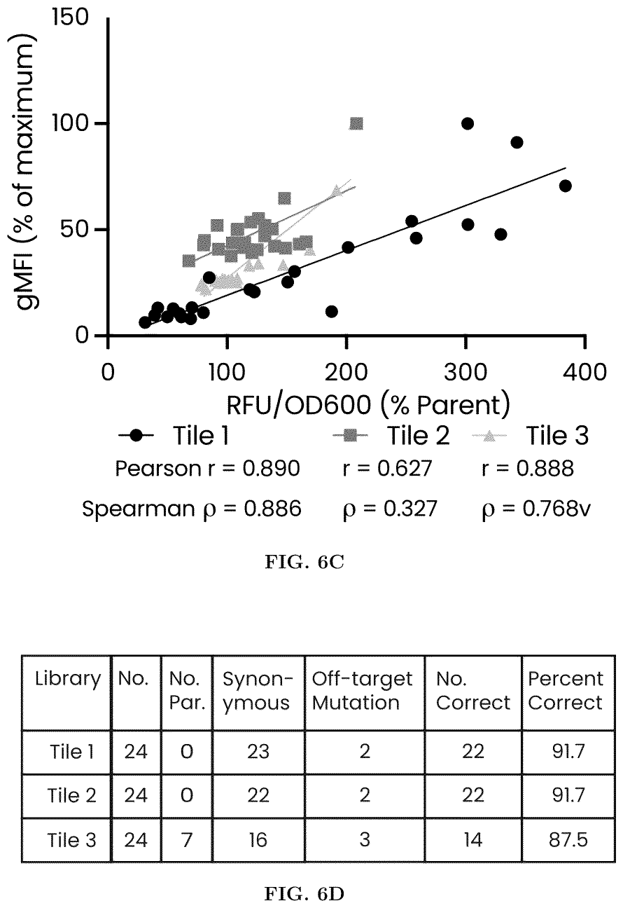

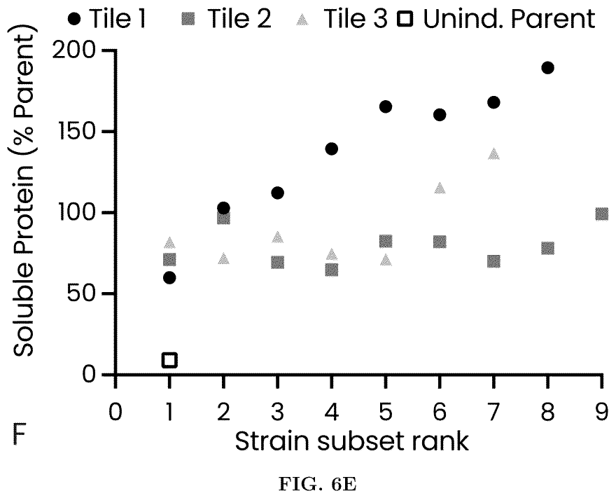

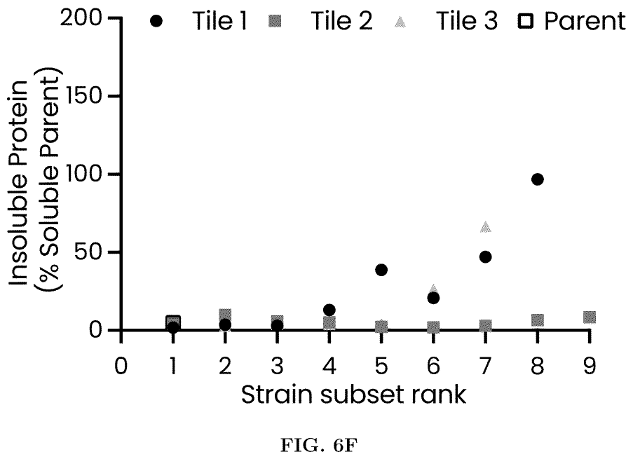

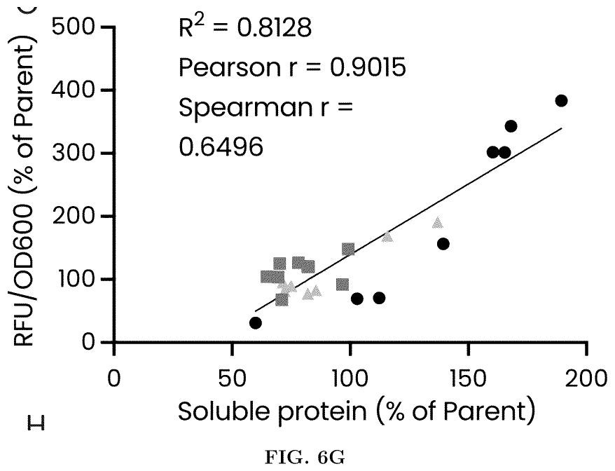

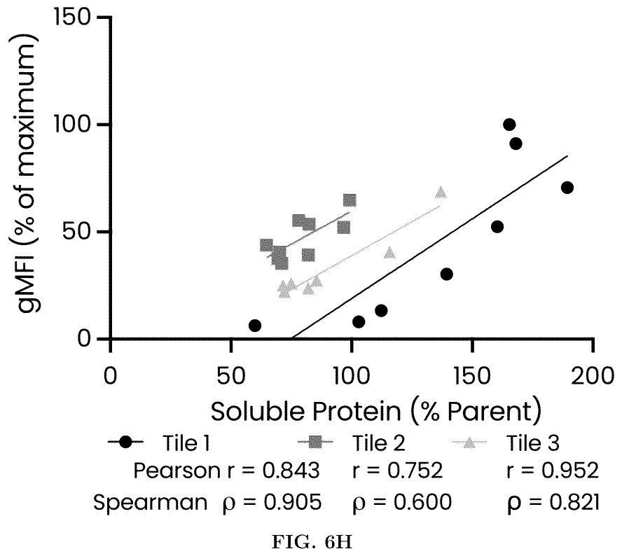

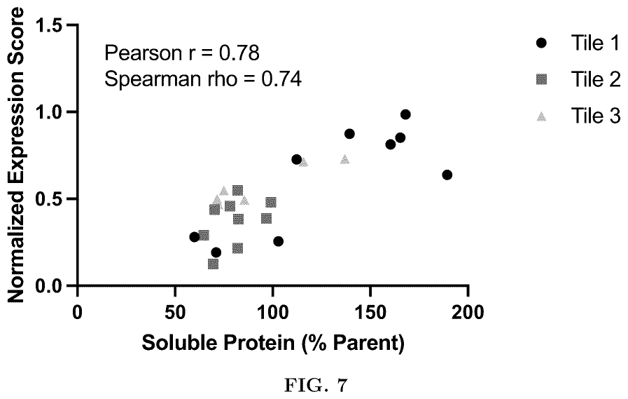

Sort-seq expression measurements were normalized across the libraries (FIG. 3N, FIG. 3O, FIG. 6A-FIG. 6H,) and scaled from 0 to 1, referred to as normalized expression scores. The present techniques observed strong correlation (Pearson=0.78) between normalized expression scores from Sort-seq and Western Blots of soluble protein for a subset of mutants (FIG. 6A-FIG. 6H and FIG. 7). Altogether, the synonymous GFP library included 119,669 measurements of unique CDSs after filtering (Table 2). The distribution of expression levels across these sequences varied according to the position of the tiles, with the first tile, spanning the initial 5′ region of the GFP CDS, having the largest dynamic range and the highest-expressing sequences. This is consistent with previous observations that codon variance in the 5′ region of a given CDS typically has the largest effect on protein expression compared to other regions. The functional GFP data was then used to fine-tune a pre-trained CO-BERTa model.

The present techniques evaluated its predictive performance, and observed a high correlation between predicted and measured expression levels (FIG. 2C; Spearman=0.854). Next, the present techniques fine-tuned the same pre-trained CO-BERTa model on a capped dataset which holds out the highest 10 measured GFP sequences. The present techniques did this to test whether the model could properly generalize to high expression level sequences lying outside of the training distribution. This capped model predicted expression scores similar to the maximal values observed in the training set (FIG. 2D), indicating the model's ability to accurately rank unseen sequences, even if they are outside the range of previously observed variants. Additionally, the present techniques used the model to score the GFP variants from FIG. 1E and observed a correlation between measured fluorescence and predicted expression score (FIG. 1F,FIG. 1G; Pearson r=0.634), further demonstrating the model's ability to properly score out of distribution CDSs. This ranking ability can enable prioritization of high expressors for in vivo testing.

TABLE 1
Parent Sequences of Synonymous Variants.

| Protein | Amino Acid | DNA |
|---|---|---|
| GFP | MSKGEELFTGVVPILVELD | ATGTCTAAGGGCGAAGAG |
| | GDVNGHKFSVSGEGEGDAT | CTTTTCACGGGCGTCGTT |
| | YGKLTLKFICTTGKLPVPW | CCTATCCTGGTAGAGTTG |
| | PTLVTTFAYGLQCFARYPD | GACGGTGATGTGAATGGC |
| | HMKQHDFFKSAMPEGYVQ | CACAAATTTTCTGTGTCT |
| | ERTIFFKDDGNYKTRAEVK | GGCGAGGGTGAGGGCGAT |
| | FEGDTLVNRIELKGIDFKED | GCCACTTATGGGAAATTG |
| | GNILGHKLEYNYNSHNVYI | ACTTTAAAATTTATTTGC |
| | MADKQKNGIKVNFKIRHNI | ACAACAGGTAAATTACCT |
| | EDGSVQLADHYQQNTPIGD | GTGCCTTGGCCTACTCTG |
| | GPVLLPDNHYLSTQSALSKD | GTTACGACTTTCGCGTAT |
| | PNEKRDHMVLLEFVTAAGI | GGGCTGCAATGTTTTGCC |
| | THGMDELYK* | CGGTATCCTGACCATATG |
| | | AAGCAGCATGACTTCTTT |
| | | AAGAGCGCGATGCCAGAA |
| | | GGTTACGTGCAGGAACGC |
| | | ACTATCTTCTTTAAAGAC |
| | | GACGGGAATTATAAAACT |
| | | CGGGGGGAGGTAAAATTT |
| | | GAGGGGGACACCCTTGTC |
| | | AACCGCATCGAATTGAAG |
| | | GGGATCGACTTCAAAGAG |
| | | GACGGCAACATTCTTGGG |
| | | CATAAGCTCGAATACAAT |
| | | TACAACTCGCACAATGTC |
| | | TACATTATGGCTGATAAA |
| | | CAGAAAAACGGTATCAAG |
| | | GTAAATTTCAAGATTCGC |
| | | CATAATATCGAGGATGGT |
| | | TCAGTCCAGTTGGCTGAC |
| | | CACTACCAGCAGAATACA |
| | | CCTATCGGGGACGGCCCA |
| | | GTGTTACTTCCTGACAAC |
| | | CATTACTTATCGACTCAG |
| | | TCTGCTCTGTCTAAAGAC |
| | | CCAAATGAAAAGCGGGAC |
| | | CACATGGTACTGCTGGAA |
| | | TTTGTTACCGCCGCAGGG |
| | | ATTACACACGGGATGGAT |
| | | GAATTGTATAAGTAA |
| folA | MISLIAALAVDRVIGMENAM | ATGATTAGTTTAATTGCT |
| | PWNLPADLAWFKRNTLNK | GCTCTGGCGGTGGATCGC |
| | PVIMGRHTWESIGRPLPGR | GTGATTGGAATGGAAAAC |
| | KNIILSSQPGTDDRVTWVK | GCTATGCCCTGGAATTTA |
| | SVDEAIAACGDVPEIMVIGG | CCGGCGGACCTGGCTTGG |
| | GRVYEQFLPKAQKLYLTHI | TTCAAGCGCAATACGTTG |
| | DAEVEGDTHFPDYEPDDW | AATAAACCTGTTATTATG |
| | ESVFSEFHDADAQNSHSYCF | GGACGCCATACCTGGGAG |
| | EILERR* | TCTATTGGACGCCCCCTT |
| | | CCGGGCCGCAAAAACATT |
| | | ATTCTTTCGTCACAACCT |
| | | GGCACTGATGATCGCGTG |
| | | ACATGGGTAAAGAGTGTC |
| | | GATGAAGCTATCGCCGCC |
| | | TGTGGCGATGTCCCAGAA |
| | | ATTATGGTAATTGGCGGA |
| | | GGGCGCGTCTACGAGCAA |
| | | TTCCTTCCTAAGGCCCAG |
| | | AAACTGTACTTGACACAT |
| | | ATTGATGCCGAGGTTGAA |
| | | GGTGACACCCACTTCCCA |
| | | GATTACGAACCGGATGAT |
| | | TGGGAAAGCGTTTTTAGT |
| | | GAATTTCACGACGCCGAT |
| | | GCCCAGAACTCTCACTCC |
| | | TATTGTTTCGAAATCTTA |
| | | GAGCGTCGTTAA |
| Anti- | MEVQLVESGGGLVQAGGSL | ATGGAAGTACAATTAGTA |
| HER2 | RLSCATSGITFMRYALGWY | GAATCAGGGGGGGGGTTA |
| VHH | RQSPGKQREMVASINSGGT | GTCCAGGCTGGGGGGTCG |
| | TNYADSVKGRFTISRDNAK | TTGCGGTTGTCTTGTGCC |
| | NTVYLQMNSLKPEDTAVYY | ACCTCCGGGATTACTTTC |
| | CNARWVKPQFIDNNYWGQ | ATGCGTTATGCTTTGGGC |
| | GTQVTVSSHHHHHH* | TGGTATCGCCAATCGCCT |
| | | GGTAAACAGCGTGAGATG |
| | | GTGGCATCAATTAATAGC |
| | | GGTGGCACAACGAACTAC |
| | | GCTGATAGTGTTAAAGGT |
| | | CGTTTCACCATCAGTCGG |
| | | GATAACGCAAAAAATACC |
| | | GTGTACTTGCAGATGAAC |
| | | TCCTTGAAGCCTGAAGAT |
| | | ACTGCTGTCTATTACTGC |
| | | AATGCGCGGTGGGTGAAG |
| | | CCACAGTTTATTGATAAC |
| | | AACTATTGGGGTCAGGGG |
| | | ACTCAGGTGACGGTCAGC |
| | | AGCCACCATCACCACCAT |
| | | CATTAA |
| SARS- | MQVQLVESGGGLVQPGGSL | ATGCAAGTACAGTTAGTA |
| COV-2 | RLSCAASGITLDYYAIGWFR | GAGAGTGGTGGCGGTCTC |
| VHH | QAPGKEREGVSRIRSSDGST | GTACAACCAGGGGGCTCT |
| | NYADSVKGRFTMSRDNAK | CTCCGTTTAAGTTGTGCC |
| | NTVYLQMNSLKPEDTAVYY | GCTTCGGGTATTACGTTG |
| | CAYGPLTKYGSSWYWPYE | GATTATTATGCCATTGGT |
| | YDYWGQGTQVTVSSHHHH | TGGTTTCGGCAGGCACCT |
| | HH* | GGGAAGGAGCGGGAGGGT |
| | | GTCAGCCGTATCCGGTCA |
| | | TCCGATGGGTCTACCAAT |
| | | TATGCCGATAGCGTCAAA |
| | | GGTCGTTTTACCATGTCG |
| | | CGTGATAACGCAAAGAAC |
| | | ACAGTATACCTTCAAATG |
| | | AACTCATTAAAACCAGAA |
| | | GATACGGCTGTCTACTAT |
| | | TGTGCATATGGTCCACTG |
| | | ACGAAATATGGCTCAAGT |
| | | TGGTATTGGCCTTACGAA |
| | | TACGACTACTGGGGCCAA |
| | | GGGACGCAAGTAACTGTT |
| | | AGTAGCCACCATCACCAC |
| | | CATCATTAA |
| mCherry | MVSKGEEDNMAIIKEFMRF | ATGGTCAGCAAAGGCGAA |
| | KVHMEGSVNGHEFEIEGEG | GAGGACAATATGGCAATC |
| | EGRPYEGTQTAKLKVTKG | ATTAAAGAATTTATGCGT |
| | GPLPFAWDILSPQFMYGSK | TTCAAGGTGCACATGGAA |
| | AYVKHPADIPDYLKLSFPEG | GGTAGCGTTAACGGTCAC |
| | FKWERVMNFEDGGVVTVT | GAATTTGAGATTGAGGGT |
| | QDSSLQDGEFIYKVKLRGT | GAGGGTGAAGGTCGCCCG |
| | NFPSDGPVMQKKTMGWEA | TATGAAGGCACCCAAACC |
| | SSERMYPEDGALKGEIKQR | GCGAAACTGAAGGTCACC |
| | LKLKDGGHYDAEVKTTYK | AAGGGTGGCCCGCTGCCG |
| | AKKPVQLPGAYNVNIKLDI | TTTGCCTGGGACATTCTG |
| | TSHNEDYTIVEQYERAEGR | AGCCCGCAGTTTATGTAC |
| | HSTGGMDELYK* | GGTAGCAAGGCGTACGTT |
| | | AAGCATCCGGCAGATATT |
| | | CCGGACTATCTGAAGCTG |
| | | TCGTTCCCTGAGGGTTTC |
| | | AAATGGGAGCGTGTGATG |
| | | AATTTCGAAGATGGCGGC |
| | | GTCGTTACGGTGACCCAA |
| | | GACTCTAGCCTGCAAGAC |
| | | GGCGAGTTCATCTACAAA |
| | | GTCAAACTGCGCGGTACC |
| | | AACTTTCCAAGCGATGGT |
| | | CCGGTTATGCAGAAGAAA |
| | | ACGATGGGTTGGGAAGCG |
| | | TCCAGCGAGCGTATGTAT |
| | | CCGGAAGATGGCGCGTTG |
| | | AAGGGTGAGATCAAACAA |
| | | CGTCTGAAGCTGAAAGAC |
| | | GGCGGCCACTATGATGCG |
| | | GAAGTGAAAACCACTTAC |
| | | AAGGCTAAGAAACCGGTG |
| | | CAGTTGCCGGGTGCATAC |
| | | AATGTTAACATCAAGCTG |
| | | GACATTACCTCCCATAAC |
| | | GAAGATTATACGATCGTT |
| | | GAGCAGTACGAGCGTGCC |
| | | GAGGGTCGCCACAGCACG |
| | | GGCGGTATGGACGAGCTG |
| | | TACAAATAA |

This strategy may generate highly diverse DNA sequences, as shown in FIG. 2F-FIG. 2H.

### Gating Schemes for Library Sorting.

In general, libraries may be sorted on FACSymphony S6 (BD Biosciences) instruments. Immediately prior to screening, 50 μL prepped sample may be transferred to a flow tube containing 1 mL PBS+3 μL propidium iodide. Aggregates, debris, and impermeable cells may be were removed with singlets, size, and PI+parent gating. The SARS-COV-2 VHH strains were screened on a Sony ID7000 spectral analyzer. Anti-Her2 VHH libraries were sorted using FACSymphony S6 (BD Biosciences) instruments. Collection gates were drawn to evenly fraction the log range of probe signal (FIG. 2J,FIG. 2K). The pooled VHH library tiles were sorted twice sequentially on the same instrument. Collection gates may be drawn to evenly fraction the log range of probe signal, as shown in FIG. 2I and FIG. 2J. Libraries may be sorted twice either sequentially on the same instrument or simultaneously on a second instrument with photomultipliers adjusted to normalize fluorescence intensity. The collected events may be processed independently as technical replicates. For both GFP and VHH libraries, six collection gates were drawn to sample across the range of fluorescence distribution of cells expressing functional protein, in some aspects. With respect to FIG. 2I, propidium iodide (PI) may be used to exclude dead cells, and collection gates are shown for each tile.

### Exemplary Modeling to Predict Expression of GFP Synonymous Mutants

In some aspects, the GFP dataset may next be used to fine-tune an NLP model for sequence to expression prediction. In some aspects, a ROBERTa model may be used, wherein the model is pre-trained on Enterobacterales CDS sequence, for expression level fine-tuning.

Empirical testing has shown high predictive ability of the model after training, as depicted in FIG. 2C. Pre-training with Enterobacterales coding sequences showed slightly higher predictive ability compared to a random weight initialization baseline (baseline supplement). Next, a version of the model was trained on a capped dataset, where the 10% highest measured GFP expressing sequences were omitted to test whether the model could properly predict expression levels of sequences outside of the training distribution. The capped model had reduced accuracy predicting the true normalized expression level, but predicted expression scores similar to the maximal values observed in the training set (FIG. 2D). Indicating the model can properly rank unseen sequences and enable prioritization of in vivo testing on high expressing variants. FIG. 2E depicts a density heatmap of expression scores between replicate sorts for the three GFP library tiles, wherein red lines indicate the parent GFP log-MFI. Pearson R correlation coefficients and number of datapoints passing quality control filters are shown for each tile.

Degenerate Codon folA Libraries

In some aspects, the present techniques may include using the E. coli dihydrofolate reductase (DHFR) gene, folA for generating synonymous mutant expression measurements. DHFR is a small monomeric enzyme that catalyzes production of tetrahydrofolate from dihydrofolate, and its overexpression confers resistance to drugs inhibiting the folate biosynthesis pathway [44, 45].

folA's relatively short coding sequence enabled the use of degenerate oligos to construct a synonymous codon library spanning the entire CDS leading to a highly sequence diverse library, as shown in, for example, in FIG. 2G, that was bottle-necked into several sub-libraries.

In some aspects, the present techniques may include using sulfamethoxazole (SMX) and trimethoprim (TMP), synergistic folate biosynthesis inhibitors [46], to select for variants with high expression of DHFR. Empirical testing showed a reduction in cell density at increased levels of antibiotic, as shown in FIG. 3A, coupled with an increased amount of soluble DHFR production, as shown in FIG. 3B.

The present techniques may include sequencing resulting post selection folA variants to calculate the frequency of synonymous mutants at increasing concentrations of antibiotic and computed a weighted average expression score based on sequence prevalence, subjected to score quality filtering, as discussed in the next section.

### Sequence Processing

To convert sequence counts from sorting and selection procedures, the following processing and quality control steps were performed:
1. Adapter sequences were removed using the CutAdapt tool.
2. Sequencing reads were merged using Fastp [cite Fastp] with the maximum overlap set according to the amplicon size and the read length used in each experiment.
3. Primer sequences were removed from both ends of merged reads using the CutAdapt tool, and reads without the primer sequences were discarded.
4. Raw counts and PPM counts were calculated for each variant.

### High-Throughput Dataset Sequencing Count Based Expression Score Calculation

To compute expression scores for the three protein datasets, the following processing and quality control steps may be performed:
1. Variants were filtered to remove DNA sequences that did not translate to the correct sequence of the target protein region.
2. The total count across all gates or antibiotic concentrations was computed for each replicate. Variants with fewer than 10 total counts in either replicate were discarded.
3. For each gate or antibiotic concentration, the variant counts were normalized by the total count (in millions).
4. The expression score for each variant in the Sort-seq and ACE experiments were computed using a weighted average using the log-transformed geometric mean fluorescence intensity within each gate (log₁₀ MFI_{gate}):

$$Expression Score = \frac{{\sum }_{gates} {count}_{gate} * {log}_{10} M F {I}_{gate}}{{\sum }_{gates} {count}_{gate}}$$

The following weighted average was used for the antibiotic selection experiment:

$$Expression Score = \frac{{\sum }_{k = 1}^{8} {count}_{k} * k}{{\sum }_{k = 1}^{8} {count}_{k}}$$

Where k is the integer rank of the antibiotic concentration (i.e. k=1 represents the lowest concentration whereas k=8 represents the highest concentration).
5. Expression scores were averaged across independent FACS sorts or replicates of antibiotic selection.
6. Specifically for the GFP data, GFP tile 1 was measured separately from GFP tiles 2 and 3. In order to reconcile batch variability in measurements, the following normalization procedure was performed. During the FACS sort, 10 sequences per tile were included as spike-in controls. The fluorescence of all 30 spike-in variants were measured alongside the tiled libraries using an FACS Symphony S6. Linear regression was performed on the log transformed mean fluorescent intensity (log-MFI) of the spike-ins to determine a scaling function that could translate log-MFI values from the tile 1 distribution to the tile ⅔ distributions (FIG. 3N). This function was applied to the expression scores in tile 1, resulting in a consistent expression score for the parent GFP sequence (FIG. 3O).

Again, the present techniques may include normalizing and scaling the weighted average expression scores from 0 to 1. Replicate selection experiments (N=4) for sub-libraries were highly correlated. FIG. 3F. During data processing, any non-synonymous mutants of DHFR may be observed and filtered out, including those known to increase enzymatic activity, as shown in Table 2. See also [48].

TABLE 2
Processing of Sequence Data for Expression Scores

| | | Raw | Correct AA | | | |
|---|---|---|---|---|---|---|
| Dataset | | Sequences | sequence | | Over 10 reads | |
| Target | Library | # | # | % | # | % |
| GFP | tile 1 | 1,318,099 | 862,168 | 65% | 42,480 | 3.22% |
| GFP | tile 2 | 1,587,238 | 1,074,099 | 68% | 45,152 | 2.84% |
| GFP | tile 3 | 452,072 | 264,379 | 58% | 28,778 | 6.37% |
| FolA | Library A | 2,870,111 | 1,170,113 | 41% | 2,601 | 0.09% |
| FolA | Library B | 3,547,929 | 1,541,237 | 43% | 3,145 | 0.09% |
| FolA | Library C | 3,028,978 | 1,152,180 | 38% | 3,092 | 0.10% |
| FolA | Library D | 4,324,141 | 1,903,028 | 44% | 3,811 | 0.09% |
| FolA | Library E | 3,135,617 | 1,099,638 | 35% | 1,977 | 0.06% |
| FolA | Library F | 3,709,504 | 810,249 | 22% | 1,497 | 0.04% |
| VHH | VHH | 3,698,925 | 2,097,177 | 57% | 16,279 | 0.44% |

In some aspects, the DHFR drug selection score may be validated. For example, the present techniques may include instructions for confirming that the drug resistance functional expression score correlated with soluble expression of DHFR, using 24 strains identified in sequencing results, as shown in FIG. 3G-FIG. 3M, wherein statistics and AUC calculations may be performed in GraphPad Prism, for example. Two-way ANOVA tests may be performed in FIG. 3H, FIG. 3K, and FIG. 3L between strain groups A, B, and C where ***=p<W0.001, *=p<0.05, ns=p >0.05.

In empirical testing, the correlation between dose-response cell density area under the curve (AUC) and soluble DHFR expression for these strains was high for soluble DHFR (Pearson r=0.884), but low for insoluble DHFR (Pearson r=0.048), indicating selection for functional soluble protein. In total, the selection experiments resulted in 17,318 unique sequence variants with associated protein functional expression scores, as shown in FIG. 3C.

The present techniques may further include performing model fine-tuning on the folA functional dataset. Resulting predictions of the model strongly correlated with measurements, as shown in FIG. 3D; Pearson r=0.907. The model also performed well on out of training distribution predictions for high expressing sequences, as depicted in FIG. 3E. The top 10% hold out set predictions show a comparably lower normalized expression score than GFP (FIG. 3D), Regardless, highly expressing sequences still were predicted with values near the highest within capped 90% training set, demonstrating proper ranking of expression levels for unseen sequences by the model. These results further highlight the ability of model predicted rankings to prioritize testing of high expressing variants in cells.

### Masked-Language Models Applied to a Degenerate Codon Vhh Library for Expression Level Prediction

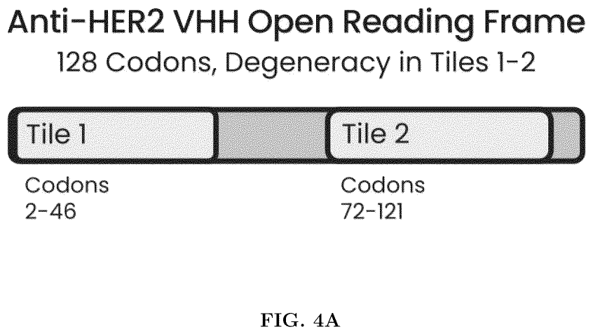

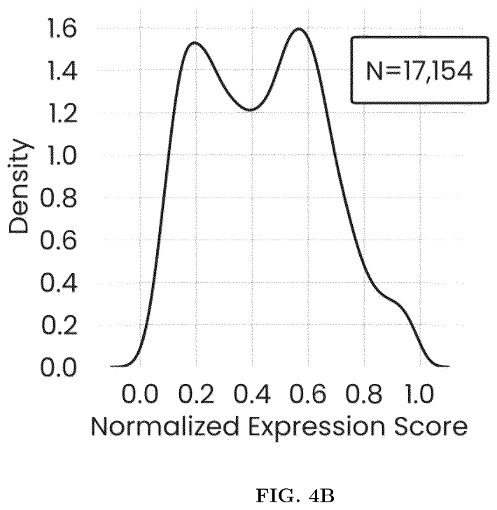

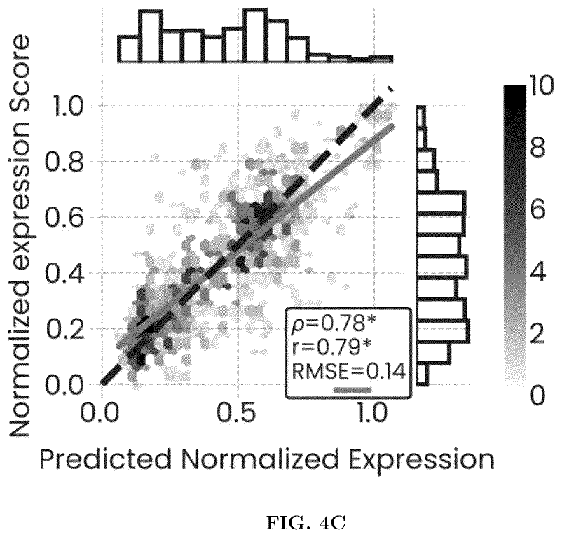

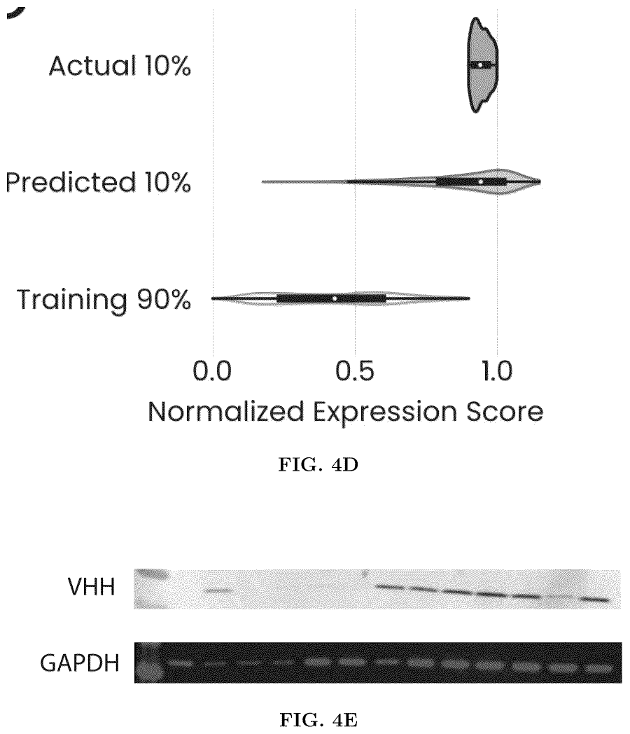

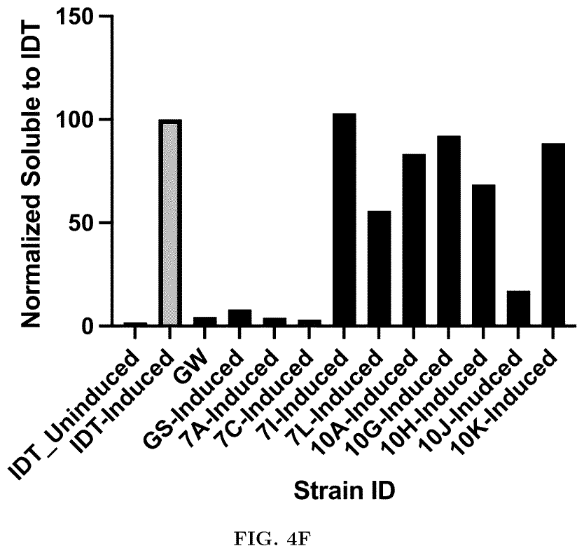

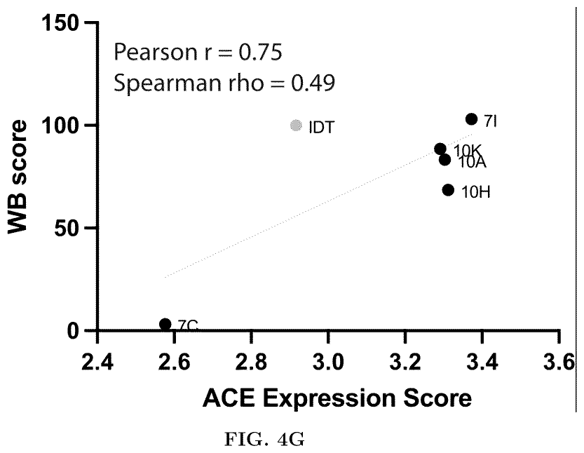

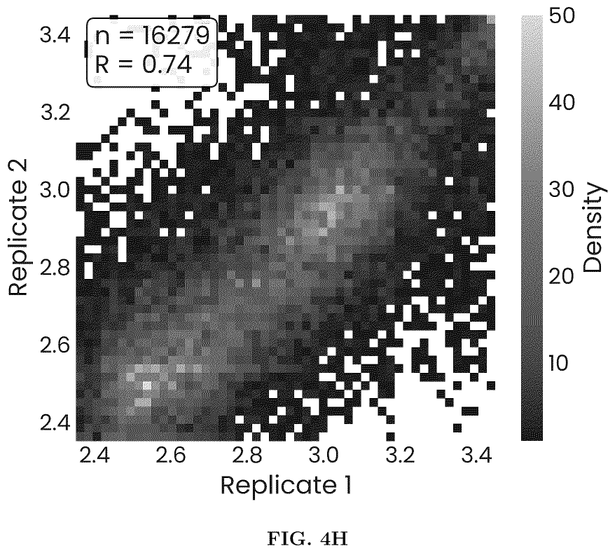

In some aspects, the present techniques may include instructions for extending the above-described synonymous codon dataset to include a protein target with relevance to industrial biotherapeutic production. For example, the present techniques may include constructing a degenerate codon library of an anti-HER2 VHH [49]. VHH-type molecules are heavy-chain-only single-domain antibodies, occurring naturally in Camelid species and can be challenging to express, properly folded in E. Coli at high levels due to the necessary disulfide bond formation. These molecules are of growing interest in biotherapeutics [51], and increasing production levels is of interest. The present techniques may include, again, instructions that apply a degenerate tile approach, where the coding parent sequence was altered with either a 5′ degenerate tile, a 3′ degenerate tile, or both degenerate tiles simultaneously, as shown in FIG. 4A. This approached generated a highly diverse library with sequence variation both in focused regions within the tiles in isolation and variation spanning the whole CDS in dual tile variants.

In some aspects, the instructions apply a version of the previously described Activity-specific Cell Enrichment (ACE) assay (FIG. 2K,FIG. 4H) to generate functional protein level measurements for VHH CDS sequence variants. The ACE assay may use antigen binding fluorescent signal as a proxy for functional protein quantity, coupled with FACS and NGS to generate expression scores for sequence library members. To validate ACE expression scores, the present techniques may include performing Western Blots on a subset of VHH sequence variants within the library. Empirical testing has shown that soluble protein levels were werll correlated with ACE expression scores, as depicted in FIG. 4E (Pearson r=0.75)-FIG. 4G, specifically validation of Anti-Her2 VHH expression scores via Western Blot.

ACE assay screening of the VHH library yielded 17,154 functional expression level measurements of unique CDS variants, as shown in FIG. 4B.

The present techniques may include fine-tuning the above-described models on the VHH dataset and assessing predictive ability similarly as with the other protein datasets. Empirical observation indicated high predictive ability of the model both for in distribution (see FIG. 4C) and out of distribution sequences (see FIG. 4D).

### Multi-Protein Learning

Advantageously, together the model performance on all three of the synonymous CDS datasets demonstrates the ability of the present language models to learn sequence to expression relationships for multiple proteins in isolation. The present techniques may include further improving model performance via multi-task learning across the three proteins. The present techniques generated a set of models trained on different combinations of the protein datasets, across either two or all three proteins. The present techniques observed an increase in model performance in all cases when training with additional proteins. Intriguingly, the present techniques also observed in some, but not all cases, that models show reasonable performance on proteins outside the training set. The best performance on this unseen protein task was observed with a model trained on folA and anti-HER2 VHH, predicting the expression of the GFP dataset. (Spearman=0.629, Pearson r=0.558). The improved accuracy in a multi-task training setting and predictive power on some unseen proteins indicates a level of generalizability in the CDS to expression level task that could be exploited to design optimized, high expressing DNA sequences. Table 5.

### Model Baseline Comparisons

To further assess the supervised models the present techniques performed a number of baseline comparisons (Table 3, Table 4). The present techniques created versions of CO-BERTa that were not pre-trained on Enterobacterales coding sequences and compared their performance to the pre-trained models from FIGS. 2-4. The present techniques find that, in almost all cases, pre-training improves accuracy slightly. Additionally, the present techniques were compared against traditional Machine Learning (ML) baselines. Specifically, the present techniques trained XGBoost models with either (1) the embeddings created by the CO-T5 model or (2) with one-hot encoded representations of the codons

Interestingly, the present techniques find similar performance for boosted tree models on individual proteins to CO-BERTa models. Furthermore, the tree-based models trained on one-hot encoded representations heavily rely on the information provided by the first codons in

TABLE 3

| Model | GFP data | folA data | VHH data |
|---|---|---|---|
| GFP full | 0.801 | 0.311 | 0.241 |
| GFP | 0.759 | 0.096 | 0.125 |
| folA | 0.347 | 0.899 | 0.428 |
| VHH | 0.137 | 0.024 | 0.782 |
| GFP + folA | 0.776 | 0.916 | 0.368 |
| GFP + VHH | 0.766 | −0.168 | 0.804 |
| folA + VHH | 0.629 | 0.905 | 0.837 |
| ALL | 0.788 | 0.918 | 0.818 |
| Pre-trained only | 0.241 | 0.116 | −0.404 |
| Finetuned only | 0.769 | 0.897 | 0.746 |
| XGBoost CO-T5 | 0.740 | 0.824 | 0.576 |
| XGBoost 1HE | 0.793 | 0.905 | 0.768 |

the sequence to predict expression values, consistent with previous findings and observations. However, training of XGBoost models is constrained to sequences of the same length and, for XGBoost models trained on CO-T5 embeddings, performance does not generalize well across different proteins (Table 6). In contrast, the DL models provide the flexibility to train and predict expression level for proteins of different length at higher accuracy. This allows for multi-protein learning which yields a boost in performance (Table 5). Additionally, the DL models can generate predictions for unseen proteins, enabling in silico design of sequences with specified protein production levels.

TABLE 4

| Model | GFP data | folA data | VHH data |
|---|---|---|---|
| GFP full | 0.854 | 0.217 | 0.181 |
| GFP | 0.808 | 0.054 | 0.057 |
| folA | 0.519 | 0.907 | 0.413 |
| VHH | 0.253 | −0.061 | 0.794 |
| GFP + folA | 0.830 | 0.910 | 0.340 |
| GFP + VHH | 0.824 | −0.127 | 0.815 |
| folA + VHH | 0.558 | 0.900 | 0.848 |
| ALL | 0.856 | 0.919 | 0.824 |
| Pre-trained only | 0.269 | 0.014 | −0.433 |
| Finetuned only | 0.835 | 0.900 | 0.745 |
| XGBoost CO-T5 | 0.760 | 0.788 | 0.555 |
| XGBoost 1HE | 0.843 | 0.898 | 0.768 |

### Multi-Protein Learning

To further assess a supervised models the present techniques performed a number of baseline comparisons, as shown in above in Table 3 and Table 4. The present techniques created versions of CO-BERTa that were not pre-trained on Enterobacterales coding sequences and compared their performance to the pre-trained models from FIGS. 2-4. The present techniques find that, in almost all cases, pre-training improves accuracy slightly. Additionally, the present techniques compare against traditional Machine Learning (ML) baselines. Specifically, the present techniques trained XGBoost models with either (1) the embeddings created by a CO-T5 model or (2) with one-hot encoded representations of the codons.

TABLE 5

| Test dataset | Training dataset | Spearman ρ | Pearson r |
|---|---|---|---|
| GFP | GFP | 0.759 | 0.808 |
| | GFP + folA | 0.776 | 0.830 |
| | GFP + VHH | 0.766 | 0.824 |
| | ALL | 0.788 | 0.856 |
| folA | folA | 0.899 | 0.907 |
| | GFP + folA | 0.916 | 0.910 |
| | folA + VHH | 0.905 | 0.900 |
| | ALL | 0.918 | 0.919 |
| VHH | VHH | 0.782 | 0.794 |
| | GFP + VHH | 0.804 | 0.815 |
| | folA + VHH | 0.837 | 0.848 |
| | ALL | 0.818 | 0.824 |

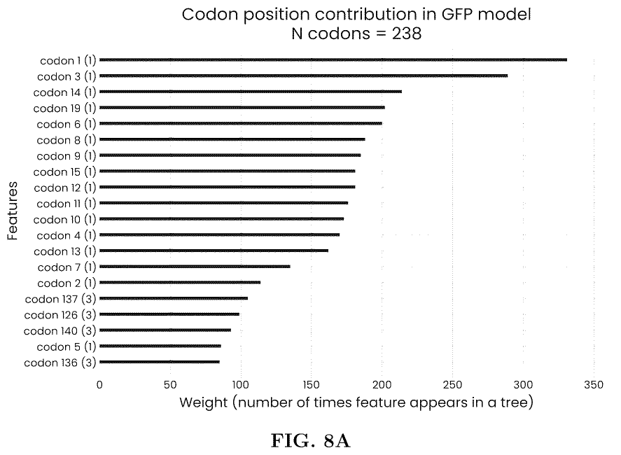

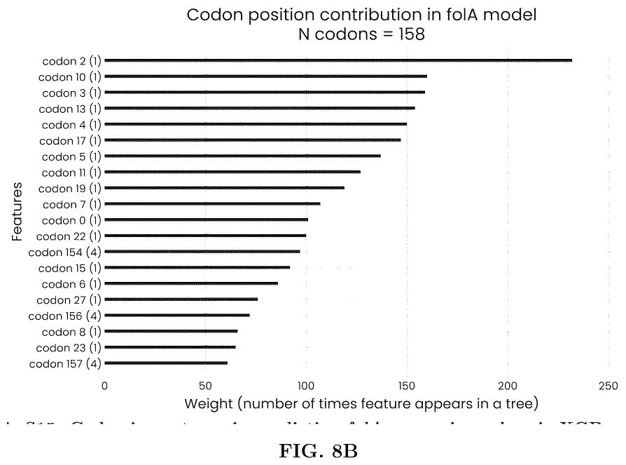

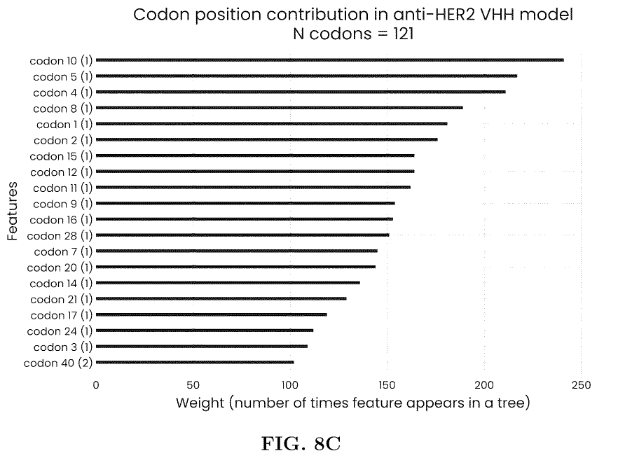

Interestingly, the present techniques find similar performance for boosted tree models on individual proteins to CO-BERTa models. Furthermore, the tree-based models trained on one-hot encoded representations heavily rely on the information provided by the first codons in the sequence to predict expression values (FIG. 8A-FIG. 8C), consistent with previous findings and observations in FIG. 2B. However, training of XGBoost models is constrained to sequences of the same length and, for XGBoost models trained on CO-T5 embeddings, performance does not generalize well across different proteins (Table 6). In contrast, the present DL models provide the flexibility to train and predict expression level for proteins of different length at higher accuracy. This allows for multi-protein learning which yields a boost in performance (Table 5). Additionally, a DL models can generate predictions for unseen proteins, enabling in silico design of sequences with specified protein production levels.

TABLE 6

| Test dataset | Training dataset | Spearman ρ | Pearson r |
|---|---|---|---|
| GFP | GFP | 0.740* | 0.761* |
| | folA | −0.144* | −0.174* |
| | VHH | −0.179* | −0.133* |
| folA | folA | 0.824* | 0.788* |
| | GFP | −0.261* | −0.221* |
| | VHH | −0.046 | −0.004 |
| VHH | VHH | 0.576* | 0.555* |
| | GFP | 0.167* | 0.155* |
| | folA | −0.127* | −0.114* |

### Multi-Protein Learning and Comparisons to Baseline Performance

In some aspects, the present techniques include instructions for training models to learn generalized sequence to expression relationships across proteins. To test this, the present techniques may include performing multi-task learning by training a model across all three of the above-described protein data sets, referred to as the ALL model.

In empirical testing, the ALL model performance improved for both folA and VHH datasets and performed equivalently for GFP predictions compared to the initial single protein model. This result demonstrated that training on new protein examples can impart models with generalized protein expression level information. To further verify model generalizability, some aspects may include generating a set of cross comparisons, testing performance across different permutations of training and test sets.

FIG. 4H depicts a density heatmap of expression scores between replicate sorts for the VHH library. Pearson R correlation coefficients and number of datapoints passing quality control filters are shown in the legend.

### Model Designed Optimized DNA Sequences on Unseen Sequences Yield High Protein Expression

Tuning Expression with Model-Designed DNA Variants for Unseen Proteins

To test the effectiveness of a model as a generalized design tool for modulating protein expression in vivo, the present techniques created a set of model-designed CDSs for two novel proteins outside the training data. The present techniques chose mCherry, a monomeric red fluorescent protein, and an anti-SARS-COV-2 VHH protein. The present techniques selected these proteins due to their modest similarity to GFP and the anti-HER2 VHH, respectively, from which the present techniques generated the synonymous variant datasets. The present techniques hypothesized the models could generalize from the given training set to related proteins. The mCherry protein sequence has 28.7% pairwise identity to GFP, while the anti-SARS-COV-2 VHH has 73.7% pairwise identity to the anti-HER2 VHH. The new proteins (mCherry and anti-SARS-COV-2 VHH) differ in amino acid length (Table 5) from their closest counterparts in the training set (GFP and anti-HER2 VHH, respectively).

Despite the low sequence identity between GFP and mCherry, the two proteins share major structural feature, namely a 8-barrel. Similarly the two VHH proteins are expected to share high structural concordance. Structural elements can influence codon usage and in turn affect protein expression and folding, potentially enabling the present model to generalized to structurally similar proteins outside the training set.

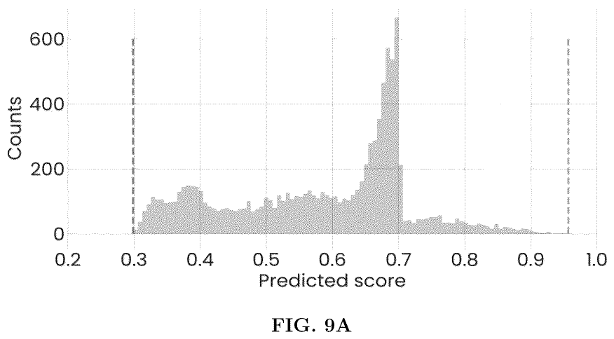

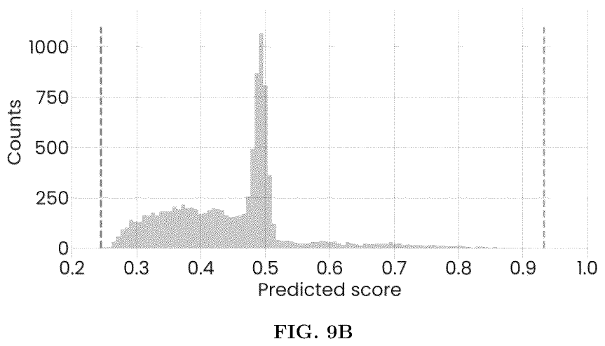

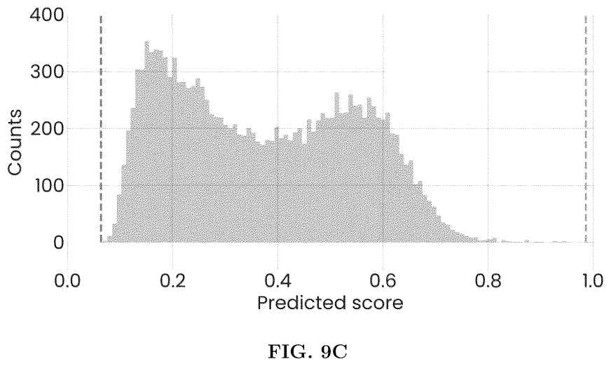

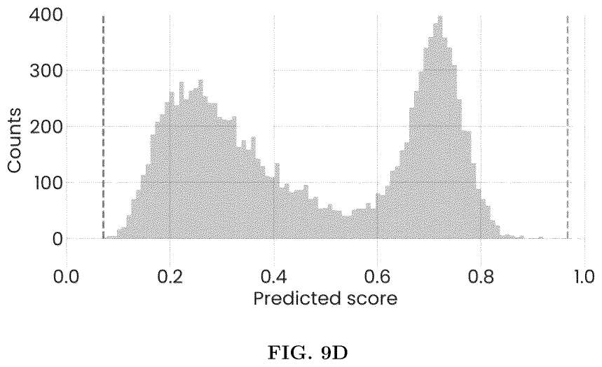

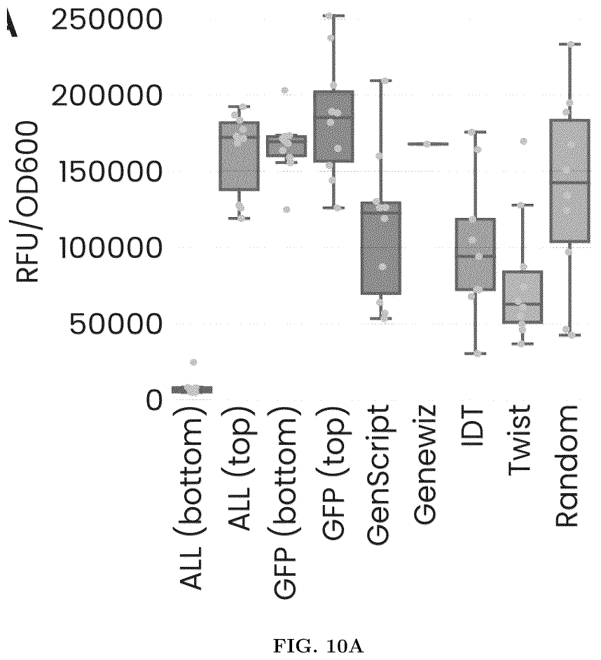

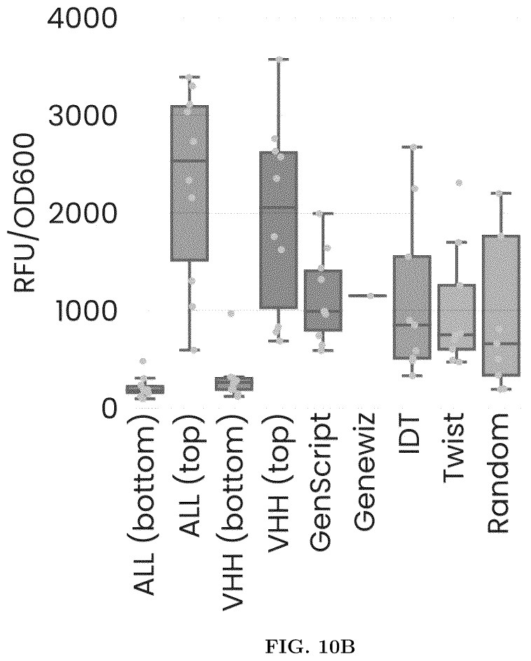

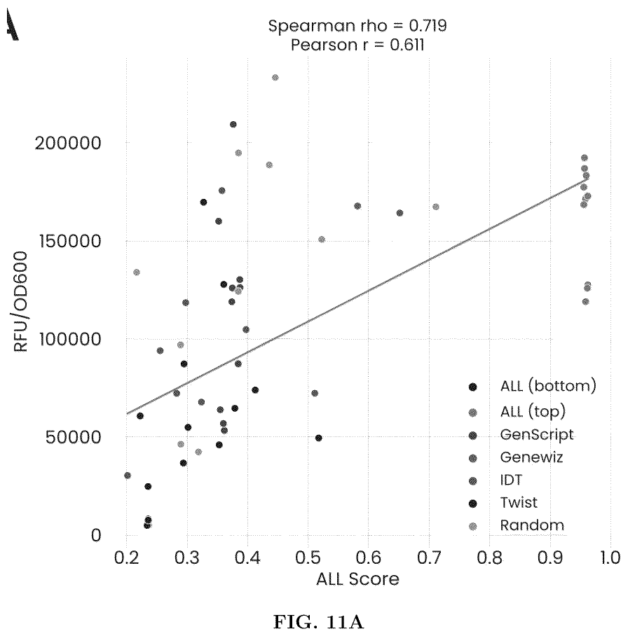

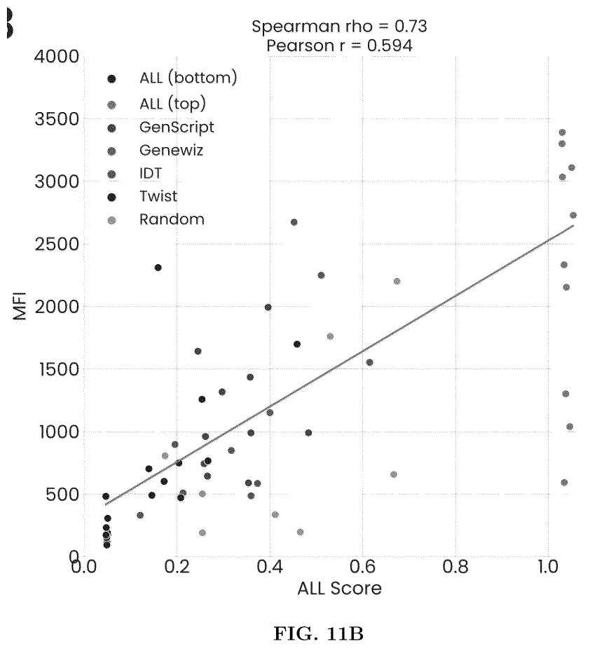

For both mCherry and the anti-SARS-COV-2 VHH, the present techniques designed CDSs with predicted high and low functional expression scores. Sequences the present techniques designed by in silico random sampling process via mutating and scoring parent CDSs in a tile scheme analogous to the GFP and anti-HER2 VHH libraries (FIG. 9B, FIG. 9C). The present techniques iteratively sampled 108 sequences and scored them with the ALL model, trained on all three protein datasets, and either the VHH or full GFP model. The 10 highest and 10 lowest scored sequences for each protein and model were selected for in vivo testing.

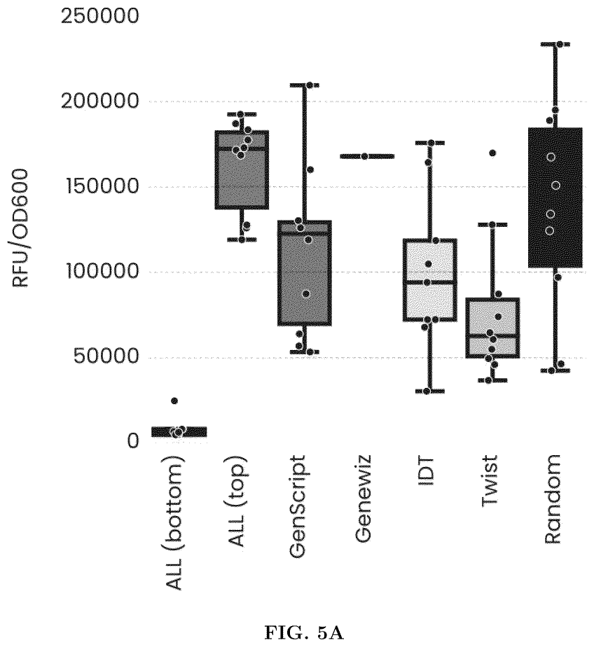

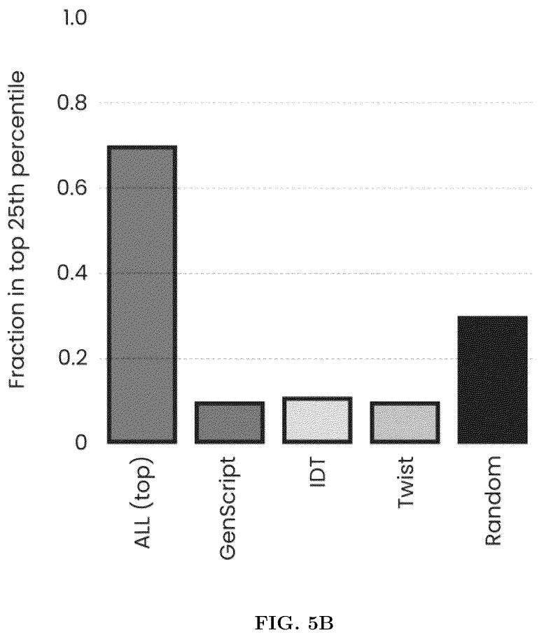

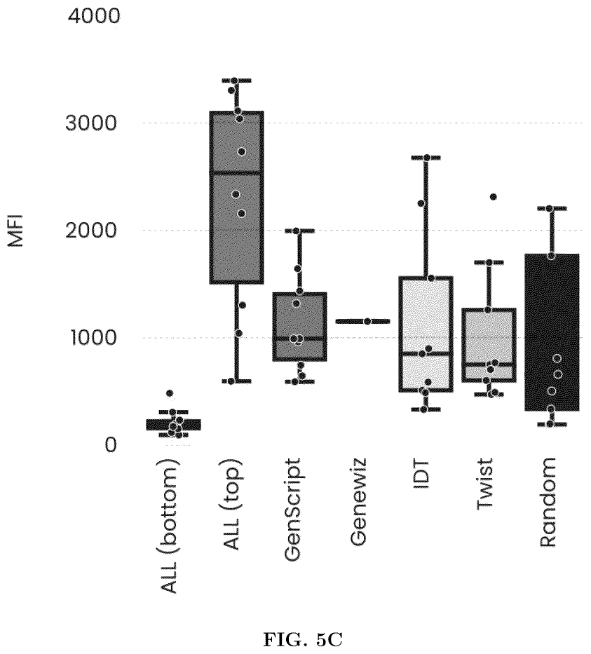

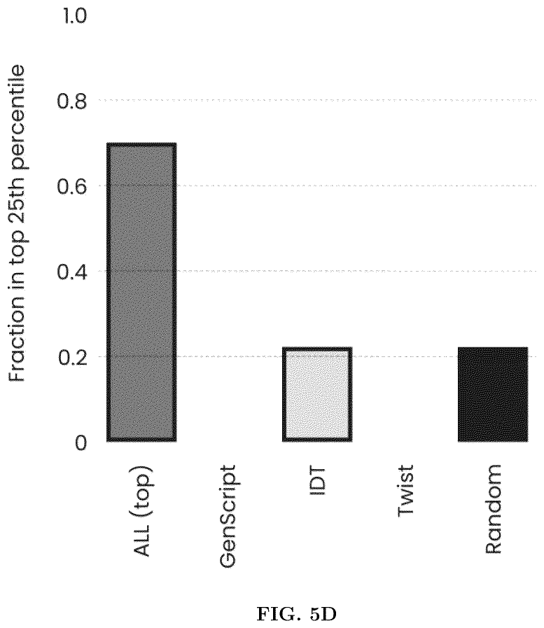

The present techniques first investigated the functional expression measurements of mCherry variants in FIG. 5A. The ALL model optimized sequences had the highest mean fluorescence among all conditions, and were significantly different by one-way ANOVA from commercial algorithms, excluding the single deterministic Genewiz sequence (p<0.05). The present techniques also observed ALL model-deoptimized sequences showed low expression, near the background of the assay.

Interestingly, GFP model-deoptimized sequences expressed relatively highly, indicating the benefit of the ALL model's multi-protein training for generalized expression level tuning of new proteins (fig. fig: FullNewDesignBoxPlot). To further illustrate the ALL model performance, the present techniques calculated the fraction of designed sequences that fell in the upper quartile of all sequences tested in FIG. 5A, except for model-deoptimized designs (FIG. 5B). Only the single genewiz sequence outperformed the ALL model by this metric.

The present techniques performed similar analyses on ACE functional expression level measurements of anti-SARS-COV-2 VHH variants. The present techniques again find the ALL model optimized CDSs showed the highest average expression and were significantly different when compared against the commercial algorithms and random sequences except for Genewiz (p<0.05) (FIG. 5C). Model deoptimized sequences once again had low expression levels. The present techniques also find the ALL model produced the highest fraction of sequences in the upper quartile of all non-deoptimized sequences (FIG. 5D). For these anti-SARS-COV-2 VHH sequences the deterministic Genewiz design did not express in the upper quartile, highlighting the unreliability of the algorithm across multiple proteins. Taken together, these design results for both proteins demonstrate the ALL model can be an effective design tool for modulating protein expression levels for new proteins outside of the training set. Additionally, the present techniques applied the ALL model to score all measured sequences in the mCherry and anti-SARS-COV-2 VHH sets in FIG. 5. The present techniques find a strong correlation between the rankings of expression level and the ALL model score (mCherry=0.72, VHH=0.73) (Supp fig). This result shows the ALL model can effectively prioritize in vivo testing of highly expressing variants, regardless of whether they the present techniques were designed using an alternative method other than the described in silico random search.

### Exemplary Computer-Implemented Methods

### Cloning

For cloning reactions, backbone fragments may be generated by PCR using proof-reading polymerase (Phusion™, ThermoFisher Cat #F530 or Q58, NEB Cat #M0492). Discrete plasmids may be constructed using the HiFi DNA Assembly kit (NEB, cat #E2621) to insert synthetic genes (IDT gBlocks or eBlocks) or isolated as single clones from libraries. All plasmids were verified by sequencing. All thermal cycling conditions are given in supplemental oligo file. Where DNA sequences may be optimized using online algorithms, the organism was chosen that was closest to E. coli strain B. For IDT, this was Escherichia coli B; for Genewiz, GenScript, and Twist, this was Escherichia coli. All predictions from all algorithms may be screened for AsiSI, AscI, BsaI, and BbsI restriction sites. Where optimizers are non-deterministic, the first optimized sequences returned may be used with no further filtering.

### Degenerate GFP Libraries

A number (e.g., four) of regions of the recombinant green fluorescent protein nucleotide sequence (GFP) may be chosen for investigation as degenerate libraries (codons 2-20, 120-150, 200-220). Backbone fragments may be amplified in two pieces, reactions may be treated with DpnI (37° C., 15 min), and amplicons may be gel-purified (QIaquick Gel Extraction Kit, Qiagen cat #28706) followed by purification (DNA Clean & Concentrator, Zymo Research cat #D4004). Libraries may be assembled using the HiFi DNA Assembly kit (NEB, cat #E2621) from degenerate Ultramer™ oligos (IDT). Reactions may be assembled with 15 μmol of each backbone fragment and 75 μmol of insert in 20 μL. For amino acids with six synonymous codons (leucine, arginine, serine) only the wobble/third position of the codon was varied from the parent sequence.

Degenerate folA Libraries

The folA gene from E. coli may be manually recoded for reconstruction with degenerate Ultramer™ oligos (IDT). A number (e.g., four) of oligos may be needed to synthesize the full degenerate gene, with junctions designed at methionine or tryptophan codons. Libraries may be constructed using scarless assembly reactions to insert oligos into plasmid backbones (BbsI-HF-v2, 1X T4 DNA ligase buffer, T4 DNA ligase; NEB). For amino acids with six synonymous codons (leucine, arginine, serine) only the wobble/third position of the codon was varied from the parent sequence. Six separate sub-libraries of approximately 10,000 variants by bottlenecking the larger library for expression level screening.

### Degenerate Anti-HER2 VHH Libraries

One or more regions of an anti-HER2 VHH sequence described in the literature

(e.g., [49]) may be chosen for investigation as degenerate libraries (e.g., codons 2-46, 72-122). To access all possible codons for amino acids with six synonymous codons (leucine, arginine, serine), sub-libraries may be constructed using oligos containing either TTR or CTN codons (leucine), TCN or AGY (serine), and CGN or AGR (arginine). These sub-libraries DNA libraries may be mixed prior to bacterial transformation. Three versions of the libraries may be constructed, a library with only 5′ gene segment degeneracy, a library with only 3′ segment degeneracy and a library with both codon tiles altered. Libraries may be assembled similarly to GFP libraries. From each of the three tile library types, approximately 10,000 variants may be mixed together to form the final VHH anti-HER2 VHH library.

### Library Bottlenecking and Strain Storage

Prepared DNA libraries and discrete plasmids may be transformed by electroporation (Bio-Rad MicroPulser) into SoluPro™ E. coli B Strain. Cells may be allowed to recover in 1 mL SOC medium for 60 min at 30° C. with 250 rpm shaking. For libraries, serial 2-fold dilutions of the recovery outgrowths may be plated on Luria broth (LB) agar with 50 μg/mL kanamycin (Teknova) and grown overnight at 37° C. Transformation efficiency was calculated, and plates with estimated colony numbers closest to our desired diversity may be harvested by scraping into LB with 50 μg/mL kanamycin. For discrete strains, single colonies may be picked into LB with 50 μg/mL kanamycin and grown overnight at 37° C. with 250 RPM shaking. For discrete strains and libraries, an equal volume of 60% sterile glycerol was added and 1 mL aliquots may be stored frozen at −80° C.

Protein Expression in E. coli

Glycerol stocks of bottlenecked libraries or discrete strains in SoluPro™ E. coli B strain may be diluted into induction base media (IBM, 4.5 g/L Potassium Phosphate monobasic, 13.8 g/L Ammonium Sulfate, 20.5 g/L yeast extract, 20.5 g/L glycerol, 1.95 g/L Citric Acid, adjusted to pH 6.8 with ammonium hydroxide) containing supplements (50 μg/mL Kanamycin, 8 mM Magnesium Sulfate, 1× Korz trace metals).

For GFP library expression, glycerol stocks containing control strains at 0.5% of cells per strain (3-4% total) may be diluted directly into 25 mL of IBM with supplements and inducers (5 μM arabinose, 5 mM proprionate) and grown for 6 hours at 30° C. with 250 rpm shaking in a baffled flask. Control strains may be grown under the same conditions as libraries in 14 mL culture tubes (4 mL volume, 250 RPM shaking) or 96 deep-well plates (1 mL volume, 1000 RPM shaking) depending on experimental need. Cultures may be immediately prepared for live-cell Sort-seq assay analysis, or harvested by centrifugation (3000 RCF, 10 min) for downstream biochemical assays.

For GFP and mCherry timecourse expression experiments, seed cultures may be created by picking single colonies from strains into 1 ml IBM culture and grown overnight at 30° C., 1000 RPM shaking. Cultures may be inoculated with seed into 200 μl IBM with inducers (5 μM arabinose, 5 mM proprionate) in clear 96-well plates at 0.1 OD and grown for 24 hours in a Biotek Synergy H1 plate reader at 30° C. Area under the curve RFU measurements normalized by OD may be collected. A one-way ANOVA statistical test may be applied to data points collected to discern statistically significant trends in the different sequence conditions tested for both mCherry and GFP variants.

For folA expression under antibiotic selection, glycerol stocks may be diluted into 50 mL IBM with supplements and grown overnight at 30° C. with 250 rpm shaking in a baffled flask. Seed cultures may be then induced (250 μM arabinose, 1 mM proprionate) and cultured for an additional 2 hours. Induced cultures may be then diluted to approximately 50,000 cells per mL in IBM with supplements and inducers (250 μM arabinose, 1 mM proprionate) and grown in 96 deep-well plates with 1 mL volume per well. Control strains may be added at a rate of 5% total cells. FolA expression libraries may be grown in the presence of sulfamethoxazole (1 μg/μL) (Research Products International, Cat #S47000) and a titration of trimethoprim (0, 1, 2, 4, 8, 16, 24, and 32 μg/mL) (Sigma Aldrich) with 0.4% dimethylsulfoxide. Plates may be grown at 30° C. with 1000 rpm shaking (3 mm throw) for 24 hours and cells may be harvested by centrifugation (3,000 RCF, 10 min) immediately prior to preparation for sequencing and downstream biochemical analyses.

For VHH expression, glycerol stocks may be diluted into LB with 50 μg/mL kanamycin and grown overnight at 37° C. with 250 rpm shaking in a baffled flask. Seed cultures may be diluted into 5 mL of IBM with supplements and inducers (250 μM arabinose, 20 mM proprionate) and grown for 22 hours at 30° C. with 250 RPM shaking. Control strains may be inoculated from glycerol stocks into 4 mL and grown as for libraries. After 22 hours growth, 1 ml aliquots of the induced culture may be adjusted to 25% v/v glycerol and stored at −80° C. before performing downstream biochemical analyses and ACE assays.

### Western Blotting

All cell cultures may be normalized to OD600=1 and centrifuged at 1500 g for 15 minutes. Cell pellets may be resuspended in 200 μL lysis buffer (1× BugBuster®) Protein Extraction Reagent, EMD Millipore; 0.025 U/μL Benzonase@ Nuclease, EMD Millipore; 1× Halt™ Pro-tease Inhibitor Cocktail, Thermo Scientific; 1 U/μL rLysozyme™, EMD Millipore), incubated at room temperature for 30 minutes, and centrifuged at 4000 g for 30 minutes. Supernatant was removed and stored as soluble material. The remaining pellet was resuspended in 200 μL lysis buffer and stored as insoluble material. Laemmli sample buffer (1× final) and dithiothreitol (DTT, 100 mM final) may be added to insoluble and soluble fractions and incubated at 70° C. for 20 min. Samples may be run on Novex WedgeWell 4-20% Tris-Glycine Gels (Invitrogen) in Tris-Glycine SDS Running Buffer (225V, 45 min) and transferred to nitrocellulose (iBlot 2, iBlot 2 NC Mini Stacks; Invitrogen) at 20 V for 1 min, 23 V for 4 min, and 25 V for 2 min. Blots may be incubated in blocking buffer (3% BSA in tris-buffered saline plus 1% Tween-20 [TBS-T]) for one hour at room temperature or 4C overnight. Quantification was performed via densitometry using AzureSpot Pro (Azure Biosystems) with rolling-ball background correction.

### GAPDH Blots

For all glyceraldehyde phosphate dehydrogenase (GAPDH) quantification, blots may be cut in half below the 35 kDA marker. The upper half was then probed with 1:1000 GAPDH Loading Control Monoclonal Antibody, Alexa Flour 647 (MA5-15738-A647, Invitrogen) in blocking buffer (1 h room temp) and imaged using an Azure600 (Cy5 fluorescence channel).

### GFP Western Blots

Blots may be incubated in 1:2000 GFP Polyclonal Antibody (A-11122, ThermoFisher Scientific) in blocking buffer (1 h, room tem), 1:2500 Goat anti-Rabbit IgG (Heavy Chain), Superclonal™ Recombinant Secondary Antibody, HRP (A27036, ThermoFisher Scientific) in blocking buffer (30 min room temp). SuperSignal PLUS Chemiluminescent substrate (ThermoFisher Scientific) was added and the membrane was incubated at room temperature for 5 minutes and imaged on an Azure300.

### DHFR Western Blots

Blots may be incubated in 1:5000 VHH against DHFR/folA (PNBL047, Creative Biolabs) in blocking buffer for (1 h room temp), 1:500 Goat anti alpaca Alexa647 (128-605-230, Jackson ImmunoResearch) in blocking buffer (30 min room temp), and imaged on an Azure600 (Cy5 fluorescence channel).

### Anti-Her2 VHH Western Blots

Blots may be incubated in 1:2000 Anti-polyhistidine-Alkaline Phosphatase antibody, Mouse monoclonal (A5588, Sigma-Aldrich) in blocking buffer (1 h room temp). 1-Step™ NBT/BCIP Substrate Solution (ThermoScientific) may be added (5 min room temp) and the membrane may be imaged on an Azure300.

### GFP Variant Measurements Via Sort-Seq Assay

To generate high-throughput expression measurements of synonymous DNA sequence variants of GFP we applied a FACS based sort-seq protocol similar to Schmitz et al. to three separate tiled degenerate libraries. For staining, aliquots of OD600=2 from induced cultures may be made in 0.7 mL matrix tubes, centrifuged at 3300 g for 3 min, and pelleted cells were washed 3× with 1×PBS+EDTA. Washed cells may be then resuspended in 500 AL of 1×PBS +EDTA for live cell sorting.

Libraries may be sorted on FACS Symphony S6. Prior to sorting, 40 μL of sample may be transferred to a FACS tube containing 1 mL PBS+EDTA with 3 μL propidium iodide. Aggregates, debris, and impermeable cells may be excluded from singlets, size, and PI+parent gates, respectively. The GFP-positive population may be divided into 6 evenly spaced gates with 400,000 events collected per gate in 3 replicates. Sorted cells may be then centrifuged for 10 minutes at 3800 g, washed once with 450 μL of DI water, resuspended, and centrifuged for 10 minutes at 3800 g. Supernatant may be aspirated and samples may be processed for DNA extraction and NGS analysis.

The three tile libraries may be screened in isolation, with tile 1 screened on a separate day than tile 2 and tile 3. Fluorescence values of the libraries may be normalized based on concurrently measured control variants, as depicted in FIG. 3N and FIG. 3O. The normalized expression score values allowed for combination of tiles into a single dataset (fig. FIG. 2B). Sequencing data from the Sort-seq method may be used to generate expression score values and subjected to quality thresholding.

FIG. 6A-FIG. 6D depict Validation of GFP degenerate codon library control variants. Specifically, Expression score values may be validated using a subset of 24 variants per tile measured via Plate Reader, Cytometer and soluble protein levels via Western Blot (see FIG. 6A-FIG. 6D), with high correlation between all metrics (see FIG. 7).

In particular, the plot of FIG. 7 shows Sort-seq derived expression scores and soluble protein levels as measured via Western Blot of representative GFP variants from FIG. 6A-FIG. 6D. Correlation is observed between the two measurement types across variants from all three tiles.

### FolA Expression Measurements Via Antibiotic Selection

Cells expressing synonymous DNA sequence variants of folA may be prepared and grown as described (cite above). Plasmid DNA may be extracted and PCR amplicons may be generated and sequenced in the described manner. Sequence counts may be used to generate weighted expression scores as described.

### VHH Variant Measurements Via Activity-Specific Cell-Enrichment (ACE) Assay

In some aspects, the present techniques may include applying a modified version of the previously described ACE assay to generate high-throughput protein level measurements of anti-Her2 and anti-SARS-COV-2 VHH synonymous DNA sequence variants expressed in SoluPro™ E. coli B strain [36].

### Cell Preparation

High-throughput screening may be performed on VHH codon libraries intracellularly stained for functional antigen binding. An OD600=2 of thawed glycerol stocks from induced cultures may be transferred to 0.7 ml matrix tubes and centrifuged at 3300 g for 3 min. The resulting pelleted cells may be washed three times with PBS+1 mM EDTA and thoroughly resuspended in 250 μL of 32 mM phosphate buffer (Na₃HPO₄) by pipetting. Cells may be fixed by the addition of 250 μL 32 mM phosphate buffer with 1.3% paraformaldehyde and 0.04% glutaraldehyde. After 40 min incubation on ice, cells may be washed three times with PBS, resuspended in lysozyme buffer (20 mM Tris, 50 mM glucose, 10 mM EDTA, 5 μg/ml lysozyme) and incubated for 8 min on ice. Fixed and lysozyme-treated cells may be equilibrated by washing 3× in stain buffer.

### Staining

After lysozyme treatment and equilibration, libraries may be the Her2 VHH library was resuspended in 500 μL Triton X-100 based stain buffer (AlphaLISA immunoassay assay buffer from Perkin Elmer; 25 mM HEPES, 0.1% casein, 1 mg/mL dextran-500, 0.5% Triton X-100, and 0.05% kathon) with 50 nM human HER2: AF647 (Acro Biosystems) and 30 nM anti-VHH probe (MonoRab anti-Camelid VHH [iFluor 488], GenScript cat #A01862). Libraries The SARS-COV-2 VHH strains were resuspended in saponin based stain buffer (1× PBS, 1 mM EDTA, 1% heat inactivated fetal bovine serum, and 0.1% saponin) with 75 nM SARS-COV-2 delta RBD: AF647 (Acro Biosytems) and 25 nM anti-VHH probe. Samples may be incubated with probe overnight (16 h) with end-to-end rotation at 4° C. protected from light. After incubation, cells may be pelleted, washed 3× with PBS, and then resuspended in 500 AL PBS by thorough pipetting.

### Amplicon Generation for NGS

Post-Selection folA Amplification

DNA may be extracted from bacterial cultures grown under selection conditions by miniprep (Qiagen, Qiaprep 96 cat #27291 or Qiaprep cat #27106). The folA variable region may be amplified by PCR (Phusion™, ThermoFisher cat #F530 or Q50, NEB cat #M0492) with 500 nM primer. See supplemental oligo file for oligo sequences and PCR conditions. PCR reactions may be then purified using ExoSAP-IT PCR Product Cleanup Reagent (ThermoFisher), quantified by Qubit fluorometer (Invitrogen), normalized, and pooled. Pool size may be verified via Tapestation 1000 HS and sequenced.

### Sort-Seq GFP and ACE Assay Anti-HER2 VHH Amplification

Cell material from various gates may be collected in a diluted PBS mixture (VWR), in 96 well plates. Post-sort samples may be spun down at 3,800 g and tube volume may be normalized to 20 μl. Amplicons for sequencing may be generated via PCR, using collected cell material directly as template with 500 nM primer concentration, Q5 2× master mix NEB) and 20 μl of sorted cell material input suspended in diluted PBS (VWR). See supplemental oligo file for oligo sequences and PCR conditions. PCR reactions may be then purified using PCR reactions may be then purified using ExoSAP-IT PCR Product Cleanup Reagent (ThermoFisher), quantified by Qubit fluorometer (Invitrogen), normalized, and pooled. Pool size may be verified via Tapestation 1000 HS and sequenced.

### Sequencing

Amplicons may be prepared for sequencing using Takara ThruPLEX® DNA-Seq Kit (Takara Bio, cat #R400674), which included unique dual indexing. To ensure a minimum of 50 bp read overlap, libraries with insert sizes of 250 bp or greater may be sequenced using 2×300 paired-end reads on an Illumina MiSeq using a MiSeq Reagent Kit v3 (Illumina Inc, MS-102-3003). Libraries with insert sizes of less than 250 bp may be sequenced using 2×150 paired-end reads. These may be sequenced on either an Illumina MiSeq or NextSeq, depending on the read depth required for each run. Each run ay be sequenced with a 20% PhiX spike-in for diversity.

### Synonymous DNA Expression Datasets

The three datasets (GFP, anti-HER2 VHH, and folA) outlined above may be used to fine-tune the pre-trained MLM CO-BERTa model as a sequence-to-expression predictor.

Each dataset was first filtered to include sequences with at least 10 read counts, resulting in the following dataset sizes: GFP=119,703 sequences, anti-HER2 VHH=17,146 sequences, and folA=17,319 sequences. These may be then used to fine-tune the pre-trained model in three different ways: (1) using the GFP dataset alone, (2) using the anti-HER2 VHH dataset alone, and (3) using sequences from all three datasets. Since the GFP dataset was significantly larger than the other two, we randomly sampled 18,000 GFP sequences for fine-tuning the model in this last case. In each case, 90% of all sequences may be used for fine-tuning and the remaining 10% may be held out as validation and test sets (5% of the dataset, respectively).

### Sequence Naturalness

The present techniques define the naturalness nₛ of a sequence as the inverse of its pseudo-perplexity. Recall that, for a sequence S with N tokens, the pseudo-likelihood that a model with parameters ⊖ assigns to this sequence is given by:

$$PLL ( S ) = {\sum }_{t = 1}^{\vert S \vert} log {P}_{MLM} ( t \vert {S}_{∖ t} ; Θ )$$

The pseudo-perplexity is obtained by first normalizing the pseudo-likelihood by the sequence length and then applying the negative exponentiation function:

$$PPPL ( S ) = exp ( - \frac{1}{\vert S \vert} PLL ( S ) )$$

Thus, the sequence naturalness is:

$${n}_{s} = \frac{1}{PPPL ( S )} = exp ( \frac{1}{\vert S \vert} PLL ( S ) )$$

Sequence metrics (CAI, GC %, and % MinMax)

For each DNA sequence generated by the pre-trained CO-T5 model, the present techniques computed three metrics as described before: Codon Adaptation Index (CAI) [38], GC % content, and % MinMax [19].

Briefly, the CAI of a DNA coding sequence with N codons is the geometric mean of the frequencies of its codons in the original source genome.

$$C A I = \sqrt[N]{{\prod }_{i = 1}^{N} {f}_{i}}$$

GC % is the fraction of nucleotides in the DNA coding sequence that are either Guanine (G) or Cytosine (C). The algorithm for computing % MinMax has been described in detail elsewhere [19]. In the present implementation, a codon window size of 18 may be used.

### Generation and Model Scoring of in Silico Degenerate Codon Libraries

Using the present fine-tuned CO-BERTa models, the present techniques scored 10 million random synonymous DNA coding variants in silico for each mCherry and anti-COVID VHH. The present techniques may restrict the insertion of random synonymous codons to the same tiles used in the GFP and anti-HER2 VHH libraries, to ensure that similarity to the datasets used during fine-tuning is maintained. For the mCherry library, the present techniques may score sequences with both the GFP and the ALL models, whereas for the anti-COVID VHH library we used the anti-HER2 and the ALL models. The present techniques may select the top 10 best and bottom 10 worst scored sequences for each library as scored by each of the two corresponding models, and randomly select 10 sequences from the rest for the downstream experimental validation.

### XGBOOST Baseline Model Training

The present techniques may train baseline XGBoost models using two different approaches. First, using the pre-trained CO-T5 model, the present techniques may generate embeddings for all sequences in the Synonymous DNA Expression Datasets (GFP, folA, and anti-HER2 VHH). The present techniques may then train three individual XGBoost models with each dataset by using a random split of 90% for training and 5% for validation and test holdout each. This first type of models were dubbed XGBoost CO-T5. For the second approach, the present techniques may train the same three XGBoost models except that each sequence was converted to a one-hot encoding by mapping each of the 64 codons to a unique value from 1 through 64. These models are dubbed XGBoost 1HE. For all models, the present techniques may the xgboost package for Python (version 1.7.2) and the xgboost.train function with the following hyperparameters: nboosts=100, eta=0.1, booster=‘gbtree’, objective=‘reg: squarederror’.

### Exemplary Computer-Implemented Method for Generalized Codon Optimization for Increased Protein Expression Via Large-Scale Synonymous DNA Variant Datasets and Deep Learning

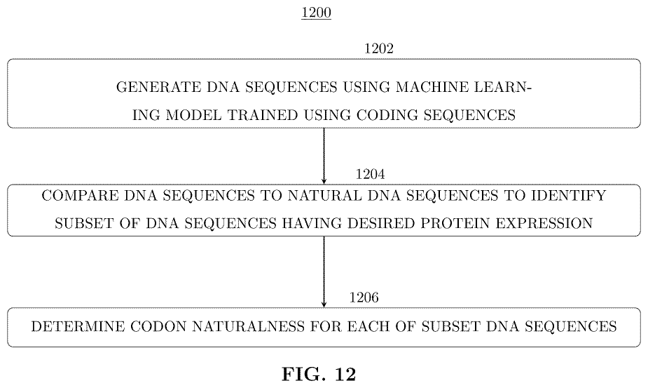

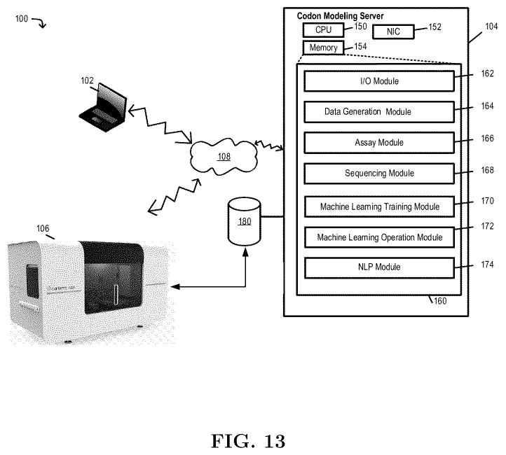

FIG. 12 depicts a block diagram of a computer-implemented method 1200, according to some aspects.

### Exemplary Computer-Implemented Machine Learning Training and Operation

FIG. 13 depicts an exemplary computing environment 100 for training and/or operating one or more machine learning (ML) models, according to some aspects. The environment 100 includes a client computing device 102, a codon modeling server 104, an assay device 106 and an electronic network 108. Some aspects may include a plurality of client devices 102, a plurality of codon modeling servers 104, and/or a plurality of assay devices 106. Generally, the one or more codon modeling servers 104 operates to perform training and operation of full or partial in silico codon modeling as described herein.

The client computing device 102 may be an individual server, a group (e.g., cluster) of multiple servers, or another suitable type of computing device or system (e.g., a collection of computing resources). For example, the client computing device 102 may be any suitable computing device (e.g., a server, a mobile computing device, a smart phone, a tablet, a laptop, a wearable device, etc.). In some aspects, one or more components of the client device 102 may be embodied by one or more virtual instances (e.g., a cloud-based virtualization service) and/or may be included in a respective remote data center (e.g., a cloud computing environment, a public cloud, a private cloud, hybrid cloud, etc.). The client computing device 102 includes a processor and a network interface controller (NIC). The processor may include any suitable number of processors and/or processor types, such as CPUs and one or more graphics processing units (GPUs). Generally, the processor is configured to execute software instructions stored in a memory. The memory may include one or more persistent memories (e.g., a hard drive/solid state memory) and stores one or more set of computer executable instructions/modules. For example, the executable instructions may receive and/or display results generated by the server 104.

The client computing device 102 may include a respective input device and a respective output device. The respective input devices may include any suitable device or devices for receiving input, such as one or more microphone, one or more camera, a hardware keyboard, a hardware mouse, a capacitive touch screen, etc. The respective output devices may include any suitable device for conveying output, such as a hardware speaker, a computer monitor, a touch screen, etc. In some cases, the input device and the output device may be integrated into a single device, such as a touch screen device that accepts user input and displays output. The NIC of the client computing device may include any suitable network interface controller(s), such as wired/wireless controllers (e.g., Ethernet controllers), and facilitate bidirectional/multiplexed networking over the network between the client computing device 102 and other components of the environment 100.

The codon modeling server 104 includes a processor 150, a network interface controller (NIC) 152 and a memory 154. The codon modeling server 104 may further include a data repository 180. The data repository 180 may be a structured query language (SQL) database (e.g., a MySQL database, an Oracle database, etc.) or another type of database (e.g., a not only SQL (NoSQL) database). In some aspects, the data repository 180 may comprise file system (e.g., an E×T filesystem, Apple file system (APFS), a networked filesystem (NFS), local filesystem, etc.), an object store (e.g., Amazon Web Services S3), a data lake, etc. The data repository 180 may include a plurality of data types, such as pretraining data sourced from public data sources (e.g., OAS data) and fine-tuning data. Fine-tuning data may be proprietary affinity data that is sourced from a quantitative assay ACE, Carterra, or any other suitable source.

The server 104 may include a library of client bindings for accessing the data repository 180. In some aspects, the data repository 180 is located remote from the codon modeling server 104. For example, the data repository 180 may be implemented using a RESTdb.IO database, an Amazon Relational Database Service (RDS), etc. in some aspects. In some aspects, the codon modeling server 104 may include a client-server platform technology such as Python, PHP, ASP .NET, Java J2EE, Ruby on Rails, Node.js, a web service or online API, responsive for receiving and responding to electronic requests. Further, the codon modeling server 104 may include sets of instructions for performing machine learning operations, as discussed below, that may be integrated with the client-server platform technology.

The assay device 106 may be a Surface Plasmon Resonance (SPR) machine, for example, such as a Carterra SPR machine. The device 106 may be physically connected to either the codon modeling server 104 or the data repository 180, as depicted. The device 106 may be located in a laboratory, and may be accessible from one or more computers within the laboratory (not depicted) and/or from the codon modeling server 104. The device 106 may generate data and upload that data to the data repository 180, directly and/or via the laboratory computer(s). The assay device 106 may include instructions for receiving one or more sequences (e.g., mutated sequences) and for synthesizing those sequences. The synthesis may sometimes be performed via another technique (e.g., via a different device or via a human). In some aspects, the device 106 may be configured not as a device, but as an alternative assay that can measure protein-protein interactions as listed in other sections of this application. For example, the device 106 may instead be configured as a suite of devices/workflows, including plates and liquid handling. In general, the device 106 may be substituted with suitable hardware and/or software optionally including human operators to generate data.

The network 108 may be a single communication network, or may include multiple communication networks of one or more types (e.g., one or more wired and/or wireless local area networks (LANs), and/or one or more wired and/or wireless wide area networks (WANs) such as the Internet). The network 108 may enable bidirectional communication between the client computing device 102 and the codon modeling server 104, for example.

The processor 150 may include any suitable number of processors and/or processor types, such as CPUs and one or more graphics processing units (GPUs). Generally, the processor 150 is configured to execute software instructions stored in the memory 154. The memory 154 may include one or more persistent memories (e.g., a hard drive/solid state memory) and stores one or more set of computer executable instructions/modules 160, including an input/output (I/O) module 162, a data generation module 164, an assay module 166, a sequencing module 168, a machine learning training module 170, a machine learning operation module 172; and an NLP module 174.

Each of the modules 160 implements specific functionality related to the present techniques, as will be described further, below. The modules 160 may store machine readable instructions, including one or more application(s), one or more software component(s), and/or one or more APIs, which may be implemented to facilitate or perform the features, functions, or other disclosure described herein, such as any methods, processes, elements or limitations, as illustrated, depicted, or described for the various flowcharts, illustrations, diagrams, figures, and/or other disclosure herein. In some aspects, a plurality of the modules 160 may act in concert implement a particular technique. For example, the machine learning operation module 172 may load information from one or more other models prior to, during and/or after initiating an inference operation. Thus, the modules 160 may exchange data via suitable techniques, e.g., via inter-process communication (IPC), a Representational State Transfer (REST) API, etc. within a single computing device, such as the codon modeling server 104. In some aspects one or more the modules 160 may be implemented in a plurality of computing devices (e.g., a plurality of servers 104). The modules 160 may exchange data among the plurality of computing devices via a network such as the network 108. The modules 160 of FIG. 1 will now be described in greater detail.

Generally, the I/O module 162 includes instructions that enable a user (e.g., an employee of the company) to access and operate the codon modeling server 104 (e.g., via the client computing device 102). For example, the employee may be a software developer who trains one or more ML models using the ML training module 170 in preparation for using the one or more trained ML models to generate outputs used in a codon modeling project. Once the one or more ML models are trained, the same user (or another) may access the codon modeling server 104 via the I/O module to cause the codon modeling process to be initiated. The I/O module 162 may include instructions for generating one or more graphical user interfaces (GUIs) (not depicted) that collect and store parameters related to codon modeling, such as a user selection of a particular reference protein, biomolecule, codon, etc. from a list stored in the data repository 180.

The data generation module 164 may include computer-executable instructions for generating data as discussed herein. on a one or more reference biomolecules.

The assay module 166 may include computer-executable instructions for retrieving/receiving data (e.g., one or more synthesized mutated variants) via the memory 154 and/or via the data repository 180, when stored) and for controlling the assay machine 106. For example, the assay module 166 may include instructions for causing the assay machine 106 to analyze the data received. The assay module 166 may store data in the data repository 180 in association with the one received data, such that another module/process (e.g., the sequencing module 168) may retrieve the stored information, along with measurements determined by the assay machine 106.

The sequencing module 168 may include computer-executable instructions for manipulating genetic sequences and for transforming data generated by the assay module 166 and its operation of the assay machine 106, in some aspects. The sequencing module 168 may store transformed assay data in a separate database table of the electronic data repository 180, for example. The sequencing module 168 may also, in some cases, include a software library for accessing third-party data sources, such as OAS.

### Exemplary Computer-Implemented Machine Learning Model Training and Model Operation

In general, a computer program or computer based product, application, or code (e.g., the model(s), such as machine learning models, or other computing instructions described herein) may be stored on a computer usable storage medium, or tangible, non-transitory computer-readable medium (e.g., standard random access memory (RAM), an optical disc, a universal serial bus (USB) drive, or the like) having such computer-readable program code or computer instructions embodied therein, wherein the computer-readable program code or computer instructions may be installed on or otherwise adapted to be executed by the processor(s) 150 (e.g., working in connection with the respective operating system in memory 154) to facilitate, implement, or perform the machine readable instructions, methods, processes, elements or limitations, as illustrated, depicted, or described for the various flowcharts, illustrations, diagrams, figures, and/or other disclosure herein. In this regard, the program code may be implemented in any desired program language, and may be implemented as machine code, assembly code, byte code, interpretable source code or the like (e.g., via Golang, Python, C, C++, C#, Objective-C, Java, Scala, ActionScript, JavaScript, HTML, CSS, XML, etc.).

In some aspects, the present techniques may be provided to third parties for access, for example, in a paid subscription model. In such cases, the modeling server 104 may receive inputs from one or more users (e.g., via a captive web portal). These inputs may include model training inputs, trained model parameters, and/or other inputs that affect the training and/or operation of models. In some aspects, these inputs to the modeling server 104 may include inputs corresponding to training data sets or information used by trained models for inference. For example, a subscription user may use the web portal or another related aspect (e.g., a mobile device) to optimize sequences using modeling techniques provided to the user and the user's device using the modeling server 104 and the environment 100.

In some aspects, the computing modules 160 may include a ML model training module 170, comprising a set of computer-executable instructions implementing machine learning training, configuration, parameterization and/or storage functionality. The ML model training module 170 may initialize, train and/or store one or more ML models, as discussed herein. The trained ML models and their weights/parameters may be stored in the data repository 180, which is accessible or otherwise communicatively coupled to the codon modeling server 104.

For example, the ML training module 170 may train one or more ML models (e.g., an artificial neural network (ANN)). One or more training data sets may be used for model training in the present techniques, as discussed herein. The input data may have a particular shape that may affect the ANN network architecture. The elements of the training data set may comprise tensors scaled to small values (e.g., in the range of (−1.0, 1.0)). In some aspects, a preprocessing layer may be included in training (and operation) which applies principal component analysis (PCA) or another technique to the input data. PCA or another dimensionality reduction technique may be applied during training to reduce dimensionality from a high number to a relatively smaller number. Reducing dimensionality may result in a substantial reduction in computational resources (e.g., memory and CPU cycles) required to train and/or analyze the input data.

In general, training an ANN may include establishing a network architecture, or topology, adding layers including activation functions for each layer (e.g., a “leaky” rectified linear unit (ReLU), softmax, hyperbolic tangent, etc.), loss function, and optimizer. In an aspect, the ANN may use different activation functions at each layer, or as between hidden layers and the output layer. A suitable optimizer may include Adam and Nadam optimizers. In an aspect, a different neural network type may be chosen (e.g., a recurrent neural network, a deep learning neural network, etc.). Training data may be divided into training, validation, and testing data. For example, 20% of the training data set may be held back for later validation and/or testing.

In that example, 80% of the training data set may be used for training. In that example, the training data set data may be shuffled before being so divided. Dividing the dataset may also be performed in a cross-validation setting, e.g., when the data set is small. Data input to the artificial neural network may be encoded in an N-dimensional tensor, array, matrix, and/or other suitable data structure. In some aspects, training may be performed by successive evaluation (e.g., looping) of the network, using labeled training samples. The process of training the ANN may cause weights, or parameters, of the ANN to be altered. The weights may be initialized to random values. The weights may be adjusted as the network is successively trained, by using one or more gradient descent algorithms, to reduce loss and to cause the values output by the network to converge to expected, or “learned”, values. In an aspect, a regression may be used which has no activation function. Therein, input data may be normalized by mean centering, and a mean squared error loss function may be used, in addition to mean absolute error, to determine the appropriate loss as well as to quantify the accuracy of the outputs.

In some aspects, the ML training module 170 may include computer-executable instructions for performing ML model pre-training, ML model fine-tuning and/or ML model self-supervised training. Model pre-training may be known as transfer learning, and may enable training of a base model that is universal, in the sense that it can be used as a common grammar for all antibody sequences, for example. The term “pretraining” may be used to describe scenarios wherein a second training may occur (i.e., when the model may be “fine-tuned”). Transfer learning refers to the ability of the model to leverage the result (weights) of a first pre-training to better initialize the second training, which may otherwise require a random initialization. The second training, i.e., fine-tuning, may be performed using proprietary affinity data as discussed herein. The technique of combining pre-training and fine-tuning advantageously boosts performance, in that the result of the training data performs better after pre-training training than when no pre-training is performed. Model fine-tuning may be performed with respect to given antibody-antigen pairs, in some aspects.

Generally, an ML model may be trained as described herein using a supervised, semi-supervised or unsupervised machine learning program or algorithm. The machine learning program or algorithm may employ a neural network, which may be a convolutional neural network, a deep learning neural network, transformer, autoencoder and/or a combined learning module or program that learns in two or more features or feature datasets (e.g., structured data, unstructured data, etc.) in a particular areas of interest. The machine learning programs or algorithms may also include natural language processing, semantic analysis, automatic reasoning, regression analysis, support vector machine (SVM) analysis, decision tree analysis, random forest analysis, K-Nearest neighbor analysis, naïve Bayes analysis, clustering, reinforcement learning, and/or other machine learning algorithms and/or techniques (e.g., generative algorithms, genetic algorithms, etc.).

In some aspects, an ML algorithm or techniques may be chosen for a particular input based on the problem set size of the input. In some aspects, the artificial intelligence and/or machine learning based algorithms may be based on, or otherwise incorporate aspects of one or more machine learning algorithms included as a library or package executed on server(s) 104. For example, libraries may include the TensorFlow based library, the Pytorch library (e.g., PyTorch Lightning), the Keras libraries, the Jax library, the HuggingFace ecosystem (e.g., transformers, datasets and/or tokenizer libraries therein), and/or the scikit-learn Python library. However, these popular open source libraries are a nicety, and are not required. The present techniques may be implemented using other frameworks/languages.

Machine learning may involve identifying and recognizing patterns in existing data (e.g., codon expression levels) in order to facilitate making predictions, classifications, and/or identifications for subsequent data (such as using the trained models to predict expression levels). Machine learning model(s), may be created and trained based upon example data (e.g., “training data”) inputs or data (which may be termed “features” and “labels”) in order to make valid and reliable predictions for new inputs. In supervised machine learning, a machine learning program operating on a server, computing device, or otherwise processor(s), may be provided with example inputs (e.g., “features”) and their associated, or observed, outputs (e.g., “labels”) in order for the machine learning program or algorithm to determine or discover rules, relationships, patterns, or otherwise machine learning “models” that map such inputs (e.g., “features”) to the outputs (e.g., labels), for example, by determining and/or assigning weights or other metrics to the model across its various feature categories. Such rules, relationships, or otherwise models may then be provided subsequent inputs in order for the model, executing on the server, computing device, or otherwise processor(s), to predict, based on the discovered rules, relationships, or model, an expected output.

For example, the ML training module 170 may analyze labeled data at an input layer of a model having a networked layer architecture (e.g., an artificial neural network, a convolutional neural network, a deep neural network, etc.) to generate ML models. The training data may be, for example, sequence variants labeled according to affinity. During training, the labeled data may be propagated through one or more connected deep layers of the ML model to establish weights of one or more nodes, or neurons, of the respective layers. Initially, the weights may be initialized to random values, and one or more suitable activation functions may be chosen for the training process, as will be appreciated by those of ordinary skill in the art. The ML training module 170 may include training a respective output layer of the one or more machine learning models. The output layer may be trained to output a prediction.

For example, the ML models trained herein are able to predict expression levels of unseen sequences by analyzing the labeled examples provided during training. In some aspects, the expression levels may be expressed as a real number (e.g., in a regression analysis). In some aspects, the expression levels may be expressed as a boolean value (e.g., in classification). In some aspects, multiple ANNs may be separately trained and/or operated. For example, an individual model may be fine-tuned (i.e., trained) based on a pre-trained model, using transfer learning.

In unsupervised or semi-supervised machine learning, the server, computing device, or otherwise processor(s), may be required to find its own structure in unlabeled example inputs, where, for example multiple training iterations are executed by the server, computing device, or otherwise processor(s) to train multiple generations of models until a satisfactory model is generated. In the present techniques, semi-supervised learning may be used, inter alia, for natural language processing purposes. Supervised learning and/or unsupervised machine learning may also comprise retraining, relearning, or otherwise updating models with new, or different, information, which may include information received, ingested, generated, or otherwise used over time. In various aspects, training the ML models herein may include generating an ensemble model comprising multiple models or sub-models, comprising models trained by the same and/or different AI algorithms, as described herein, and that are configured to operate together.

Once the model training module 170 has initialized the one or more ML models, which may be ANNs or regression networks, for example, the model training module 170 trains the ML models by inputting genome-scale CDS sequence data, for example, into the model. The trained ML model may be expected to provide accurate codon predictions and expression prediction levels as discussed herein.

The model training module 170 may divide labeled data into a respective training data set and testing data set. The model training module 170 may train the ANN using the labeled data. The model training module 170 may compute accuracy/error metrics (e.g., cross entropy) using the test data and test corresponding sets of labels. The model training module 170 may serialize the trained model and store the trained model in a database (e.g., the data repository 180). Of course, it will be appreciated by those of ordinary skill in the art that the model training module 170 may train and store more than one model.

For example, the model training module 170 may train an individual model for predicting codon usage and to generate sequences mimicking natural codon usage profiles using a genome-scale CDS sequence data, and another model for predicting protein expression levels for unseen sequence variants by training on a quantitative functional dataset, and yet another model for improving performance using multi-task learning. It should be appreciated that the structure of the network as described may differ, depending on the embodiment.

In some aspects, the computing modules 160 may include a machine learning operation module 172, comprising a set of computer-executable instructions implementing machine learning loading, configuration, initialization and/or operation functionality. The ML operation module 172 may include instructions for storing trained models (e.g., in the electronic data repository 180, as a pickled binary, etc.). Once trained, a trained ML model may be operated in inference mode, whereupon when provided with de novo input that the model has not previously been provided, the model may output one or more predictions, classifications, etc. as described herein. In an unsupervised learning aspect, a loss minimization function may be used, for example, to teach a ML model to generate output that resembles known output (i.e., ground truth exemplars).

Once the model(s) are trained by the model training module 170, the model operation module 172 may load one or more trained models (e.g., from the data repository 180). The model operation module 172 generally applies new data that the trained model has not previously analyzed to the trained model. For example, the model operation module 172 may load a serialized model, deserialize the model, and load the model into the memory 154. The model operation module 172 may load new molecular variant data that was not used to train the trained model.

For example, the new molecular data may include sequence data, etc. as described herein, encoded as input tensors. The model operation module 172 may apply the one or more input tensor(s) to the trained ML model. The model operation module 172 may receive output (e.g., tensors, feature maps, etc.) from the trained ML model. The output of the ML model may be a prediction as discussed above associated with the input sequences. In this way, the present techniques advantageously provide a means of generating and quantitatively predicting aspects of sequence variants, including expression levels, that are far more accurate and data rich than conventional industry practices.

Another advantage is that measuring these is time consuming and expensive, as it needs to be done in the lab. By using ML, the present techniques need only perform lab measurements to generate the training set, and then can predict unmeasured sequence variants in a relatively inexpensive and fast manner due to in silico performance, rather than requiring continued use of the wet lab.

The model operation module 172 may be accessed by another element of the codon modeling server 104 (e.g., a web service). For example, the ML operation module 172 may pass its output to the NLP module 174 for processing/analysis. Alternatively, the variant identification module 174 may perform operations and provide input data to one or more trained models via the ML operation module 172 and/or the electronic data repository 180. binding, or other properties as discussed herein).

The modules 160 may include further instructions for providing the one or more sequence variants of interest as an input (e.g., via an email, as a visualization such as a chart/-graph, as an element of a GUI in a computing device such as the client computing device 102, etc.). In some aspects, a user may interact with the ML model during training and/or operation using a command line tool, an Application Programming Interface (API), a software development kit (SDK), a Jupyter notebook, etc.

Regarding the modules 160, it will be appreciated by those of ordinary skill in the art that in some aspects, the software instructions comprising the module 160 may be organized differently, and more/fewer modules may be included. For example, one or more of the modules 160 may be omitted or combined. In some aspects, additional modules may be added (e.g., a localization module). In some aspects, software libraries implementing one or more modules (e.g., Python code) may be combined, such that, for example, the ML training module 170 and ML operation module 172 are a single set of executable instructions used for training and making predictions. In still further examples, the modules 160 may not include the assay module 166 and/or the sequencing module 168. For example, a laboratory computer and/or the assay device 106 may implement those modules, and/or others of the modules 160. In that case, assays and sequencing may be performed in the laboratory to generate training data that is stored in the data repository 180 and accessed by the server 104.

### Additional Considerations

The following considerations also apply to the foregoing discussion. Throughout this specification, plural instances may implement operations or structures described as a single instance. Although individual operations of one or more methods are illustrated and described as separate operations, one or more of the individual operations may be performed concurrently, and nothing requires that the operations be performed in the order illustrated. These and other variations, modifications, additions, and improvements fall within the scope of the subject matter herein.

Unless specifically stated otherwise, discussions herein using words such as “processing,” “computing,” “calculating,” “determining,” “presenting,” “displaying,” or the like may re-fer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of “a” or “an” is employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for implementing the concepts disclosed herein, through the principles disclosed herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

### BIBLIOGRAPHY

### References

[1] Puetz J, Wurm F M. Recombinant Proteins for Industrial versus Pharmaceutical Purposes: A Review of Process and Pricing. Processes. 2019; 7 (8). doi: 10.3390/pr7080476.
[2] Walsh G. Biopharmaceutical benchmarks 2018. Nature biotechnology. 2018; 36 (12): 1136-1145.
[3] Banks M, Johnson R, Giver L, Bryant G, Guo M. Industrial production of microbial protein products. Current Opinion in Biotechnology. 2022; 75:102707. doi: https://doi.org/10.1016/j.copbio.2022.102707.
[4] Rettenbacher L A, Arauzo-Aguilera K, Buscajoni L, Castillo-Corujo A, Ferrero-Bordera B, Kostopoulou A, et al. Microbial protein cell factories fight back? Trends in Biotechnology. 2022; 40 (5): 576-590. doi: https://doi.org/10.1016/j.tibtech.2021.10.003.
[5] McElwain L, Phair K, Kealey C, Brady D. Current trends in biopharmaceuticals production in Escherichia coli. Biotechnology Letters. 2022; p. 1-15.
[6] Rashid M H. Full-length recombinant antibodies from Escherichia coli: production, characterization, effector function (Fc) engineering, and clinical evaluation. mAbs. 2022; 14 (1): 2111748. doi: 10.1080/19420862.2022.2111748.
[7] Elena C, Ravasi P, Castelli ME, Peirú S, Menzella HG. Expression of codon optimized genes in microbial systems: current industrial applications and perspectives. Frontiers in microbiology. 2014; 5:21.
[8] Marschall L, Sagmeister P, Herwig C. Tunable recombinant protein expression in E. coli: promoter systems and genetic constraints. Applied microbiology and biotechnology. 2017; 101 (2): 501-512.
[9] Kosuri S, Goodman D B, Cambray G, Mutalik V K, Gao Y, Arkin A P, et al. Compos-ability of regulatory sequences controlling transcription and translation in Escherichia coli. Proceedings of the National Academy of Sciences. 2013; 110 (34): 14024-14029.
[10] Menacho-Melgar R, Ye Z, Moreb E A, Yang T, Efromson J P, Decker J S, et al. Scalable, two-stage, autoinduction of recombinant protein expression in E. coli utilizing phosphate depletion. Biotechnology and Bioengineering. 2020; 117 (9): 2715-2727.
[11] Lozano Terol G, Gallego-Jara J, Sola Martínez RA, Canovas Díaz M, de Diego Puente T. Engineering protein production by rationally choosing a carbon and nitrogen source using E. coli BL21 acetate metabolism knockout strains. Microbial cell factories. 2019; 18 (1): 1-19.
[12] Welch M, Govindarajan S, Ness J E, Villalobos A, Gurney A, Minshull J, et al. Design parameters to control synthetic gene expression in Escherichia coli. PloS one. 2009; 4 (9): e7002.
[13] Quax T E, Claassens N J, Soll D, van der Oost J. Codon bias as a means to fine-tune gene expression. Molecular cell. 2015; 59 (2): 149-161.
[14] Boël G, Letso R, Neely H, Price W N, Wong K H, Su M, et al. Codon influence on protein expression in E. coli correlates with mRNA levels. Nature. 2016; 529 (7586): 358-363.
[15] Schmitz A, Zhang F. Massively parallel gene expression variation measurement of a synonymous codon library. BMC genomics. 2021; 22 (1): 1-12.
[16] Ranaghan M J, Li J J, Laprise D M, Garvie C W. Assessing optimal: inequalities in codon optimization algorithms. BMC biology. 2021; 19 (1): 1-13.
[17] Şen A, Kargar K, Akgün E, Pnar M. Codon optimization: a mathematical programing approach. Bioinformatics. 2020; 36 (13): 4012-4020. doi: 10.1093/bioinformatics/btaa248.
[18] Karaşan O, Şen A, Tiryaki B, Cicek A E. A unifying network modeling approach for codon optimization. Bioinformatics. 2022; 38 (16): 3935-3941. doi: 10.1093/bioinformatics/btac428.
[19] Clarke IV T F, Clark P L. Rare codons cluster. PloS one. 2008; 3 (10): e3412.
[20] Chartier M, Gaudreault F, Najmanovich R. Large-scale analysis of conserved rare codon clusters suggests an involvement in co-translational molecular recognition events. Bioinformatics. 2012; 28 (11): 1438-1445.
[21] Chaney J L, Steele A, Carmichael R, Rodriguez A, Specht A T, Ngo K, et al. Widespread position-specific conservation of synonymous rare codons within coding sequences. PLOS computational biology. 2017; 13 (5): e1005531.
[22] Medina-Muñoz SG, Kushawah G, Castellano L A, Diez M, DeVore M L, Salazar M J B, et al. Crosstalk between codon optimality and cis-regulatory elements dictates mRNA stability. Genome Biology. 2021; 22 (1). doi: 10.1186/s13059-020-02251-5.
[23] Diez M, Medina-Muñoz S G, Castellano L A, da Silva Pescador G, Wu Q, Bazzini AA. iCodon customizes gene expression based on the codon composition. Scientific Reports. 2022; 12 (1). doi: 10.1038/s41598-022-15526-7.
[24] de Freitas Nascimento J, Kelly S, Sunter J, Carrington M. Codon choice directs constitutive mRNA levels in trypanosomes. eLife. 2018; 7. doi: 10.7554/elife.32467.
[25] Wu Q, Medina S G, Kushawah G, DeVore M L, Castellano L A, Hand J M, et al. Translation affects mRNA stability in a codon-dependent manner in human cells. eLife. 2019; 8. doi: 10.7554/elife.45396.
[26] Boël G, Letso R, Neely H, Price W N, Wong K H, Su M, et al. Codon influence on protein expression in E. coli correlates with mRNA levels. Nature. 2016; 529 (7586): 358-363. doi: 10.1038/nature16509.
[27] Tuller T, Waldman Y Y, Kupiec M, Ruppin E. Translation efficiency is determined by both codon bias and folding energy. Proceedings of the national academy of sciences. 2010; 107 (8): 3645-3650.
[28] Angov E, Hillier C J, Kincaid R L, Lyon J A. Heterologous protein expression is enhanced by harmonizing the codon usage frequencies of the target gene with those of the expression host. PloS one. 2008; 3 (5): e2189.
[29] Mahmud M, Kaiser M S, Hussain A, Vassanelli S. Applications of deep learning and reinforcement learning to biological data. IEEE transactions on neural networks and learning systems. 2018; 29 (6): 2063-2079.
[30] Bepler T, Berger B. Learning the protein language: Evolution, structure, and function. Cell systems. 2021; 12 (6): 654-669.
[31] Bachas S, Rakocevic G, Spencer D, Sastry A V, Haile R, Sutton J M, et al. Antibody optimization enabled by artificial intelligence predictions of binding affinity and naturalness. bioRxiv. 2022.
[32] Benegas G, Batra S S, Song Y S. DNA language models are powerful zero-shot predictors of non-coding variant effects. bioRxiv. 2022.
[33] Yang D K, Goldman S L, Weinstein E, Marks D. Generative Models for Codon Prediction and Optimization. Machine Learning in Computational Biology. 2019.
[34] Fu H, Liang Y, Zhong X, Pan Z, Huang L, Zhang H, et al. Codon optimization with deep learning to enhance protein expression. Scientific Reports. 2020; 10 (1): 1-9.
[35] Jain R, Jain A, Mauro E, LeShane K, Densmore D. ICOR: Improving codon optimization with recurrent neural networks. bioRxiv. 2021.
[36] Liu J. Activity-specific cell enrichment; Patent Publication No. WO 2021/146626, 22.07.2021.
[37] Raffel C, Shazeer N, Roberts A, Lee K, Narang S, Matena M, et al., Exploring the Limits of Transfer Learning with a Unified Text-to-Text Transformer; 2019. Available from: https://arxiv.org/abs/1910. 10683.
[38] Sharp P M, Li W H. The codon adaptation index-a measure of directional synonymous codon usage bias, and its potential applications. Nucleic acids research. 1987;15 (3): 1281-1295.
[39] Codon optimization. Available from: https://www.genewiz. com/en/Public/Services/Gene-Synthesis/Codon-Optimization.
[40] Karacay B. Using a codon optimization tool-HOW IT works and advantages: IDT; 2022. Available from: https://www.idtdna.com/pages/education/decoded/article/using-a-codon-optimization-tool-how-it-works-and-advantages-it-provides.
[41] What does the twist codon optimization tool do?: Twist Bioscience. Available from: https://www.twistbioscience.com/faq/using-your-twist-account/what-does-twist-codon-optimization-tool-do #: ~: text=The,20Twist % 20Codon % 200ptimizer % 20Tool, codon,20frequency,20of,20% 3C8%25).
[42] Fan L. Codon optimization; Patent Publication No. WO 2020/024917, 06.02.2020.
[43] Sørensen H P, Mortensen K K. Soluble expression of recombinant proteins in the cytoplasm of Escherichia coli. Microbial cell factories. 2005; 4 (1): 1-8.
[44] Rood J I, Laird A J, Williams J W. Cloning of the Escherichia coli K-12 dihydro-folate reductase gene following Mu-mediated transposition. Gene. 1980;8 (3): 255-265. doi: https://doi.org/10.1016/0378-1119 (80) 90003-7.
[45] Tennhammar-Ekman B, Sundström L, Sköld O. New observations regarding evolution of trimethoprim resistance. Journal of Antimicrobial Chemotherapy. 1986;18 (Supplement C): 67-76.
[46] Minato Y, Dawadi S, Kordus S L, Sivanandam A, Aldrich C C, Baughn A D. Mutual potenti-ation drives synergy between trimethoprim and sulfamethoxazole. Nature Communications. 2018; 9 (1): 1003. doi: 10.1038/s41467-018-03447-x.
[47] Martin M. Cutadapt removes adapter sequences from high-throughput sequencing reads. EMBnetjournal. 2011; 17 (1): 10-12. doi: 10.14806/ej.17.1.200.
[48] Bershtein S, Mu W, Shakhnovich E I. Soluble oligomerization provides a beneficial fit-ness effect on destabilizing mutations. Proceedings of the National Academy of Sciences. 2012; 109 (13): 4857-4862.
[49] Hartmann L, Botzanowski T, Galibert M, Jullian M, Chabrol E, Zeder-Lutz G, et al. VHH characterization. Comparison of recombinant with chemically synthesized anti-HER2 VHH. Protein Science. 2019; 28 (10): 1865-1879. doi: https://doi.org/10.1002/pro.3712.
[50] Asaadi Y, Jouneghani F F, Janani S, Rahbarizadeh F. A comprehensive comparison between camelid nanobodies and single chain variable fragments. Biomarker Research. 2021; 9 (1): 1-20.
[51] Arbabi-Ghahroudi M. Camelid Single-Domain Antibodies: Promises and Challenges as Lifesaving Treatments. International Journal of Molecular Sciences. 2022; 23 (9): 5009.
[52] Güttler T, Aksu M, Dickmanns A, Stegmann K M, Gregor K, Rees R, et al. Neutralization of SARS-COV-2 by highly potent, hyperthermostable, and mutation-tolerant nanobodies. The EMBO journal. 2021; 40 (19): e107985.

1. A computer-implemented method for performing generalized codon optimization for improved protein expression, the method comprising:
generating, via one or more processors, one or more DNA sequences using a machine learning model trained using coding sequences;
comparing, via one or more processors, the DNA sequences to one or more natural DNA sequences to identify a subset of DNA sequences that correspond to a protein expression within a predefined range; and
determining a codon naturalness for each of the subset of DNA sequences.

2. The computer-implemented method of claim 1, wherein determining the codon naturalness for each of the subset of DNA sequences includes computing an inverse of a loss function value of the machine learning model, or an inverse of the model's pseudo perplexity.

3. The computer-implemented method of claim 1, further comprising: training the machine learning model using data representing an expression level.

4. The computer-implemented method of claim 1, further comprising:
training the machine learning model using a score, wherein the score is applied to a sequence.

5. The computer-implemented method of claim 1, further comprising: training the machine learning model using a Protein2DNA dataset.

6. The computer-implemented method of claim 1, further comprising:
training the machine learning model by creating a dictionary mapping relevant characters or words from a Protein2DNA dataset to unique tokens.

7. The computer-implemented method of claim 1, further comprising: receiving, from a user device, one or more parameters; and
generating the one or more DNA sequences using the machine learning model based on the one or more parameters.

8. A computing system for performing generalized codon optimization for improved protein expression, comprising:
one or more processors; and one or more memories having stored thereon computer-executable instructions that, when executed by the one or more processors, cause the computing system to:
generate, via the one or more processors, one or more DNA sequences using a machine learning model trained using coding sequences;
compare, via one or more processors, the DNA sequences to one or more natural DNA sequences to identify a subset of DNA sequences that correspond to a protein expression within a predefined range; and
determine a codon naturalness for each of the subset of DNA sequences.

9. The computing system of claim 8, wherein determining the codon naturalness for each of the subset of DNA sequences includes computing an inverse of a loss function value of the machine learning model, or an inverse of the model's pseudo perplexity.

10. The computing system of claim 8, the memory having stored thereon further instructions that, when executed by the one or more processors, cause the computing system to:
train the machine learning model using data representing an expression level.

11. The computing system of claim 8, the memory having stored thereon further instructions that, when executed by the one or more processors, cause the computing system to:
train the machine learning model using a score, wherein the score is applied to a sequence.

12. The computing system of claim 8, the memory having stored thereon further instructions that, when executed by the one or more processors, cause the computing system to:
train the machine learning model using a Protein2DNA dataset.

13. The computing system of claim 8, the memory having stored thereon further instructions that, when executed by the one or more processors, cause the computing system to:
train the machine learning model by creating a dictionary mapping relevant characters or words from a Protein2DNA dataset to unique tokens.

14. The computing system of claim 8, the memory having stored thereon further instructions that, when executed by the one or more processors, cause the computing system to:
receive, from a user device, one or more parameters; and
generate the one or more DNA sequences using the machine learning model based on the one or more parameters.

15. A non-transitory computer-readable medium having stored thereon instructions that, when generate, via one or more processors, one or more DNA sequences using a machine learning model trained using coding sequences;
compare, via one or more processors, the DNA sequences to one or more natural DNA sequences to identify a subset of DNA sequences that correspond to a protein expression within a predefined range; and
determine a codon naturalness for each of the subset of DNA sequences.

16. The non-transitory computer-readable medium of claim claim 15 having stored thereon further instructions that, when executed by one or more processors, cause a computer to:
compute an inverse of a loss function value of the machine learning model, or an inverse of the model's pseudo perplexity.

17. The non-transitory computer-readable medium of claim 15 having stored thereon further instructions that, when executed by one or more processors, cause a computer to:
train the machine learning model using data representing an expression level.

18. The non-transitory computer-readable medium of claim 15 having stored thereon further instructions that, when executed by one or more processors, cause a computer to:
train the machine learning model using a score, wherein the score is applied to a sequence.

19. The non-transitory computer-readable medium of claim 15 having stored thereon further instructions that, when executed by one or more processors, cause a computer to:
train the machine learning model using a Protein2DNA dataset.

20. The non-transitory computer-readable medium of claim 15 having stored thereon further instructions that, when executed by one or more processors, cause a computer to:
receive, from a user device, one or more parameters; and
generate the one or more DNA sequences using the machine learning model based on the one or more parameters.
